(19) United States
(12) Patent Application Publication
(10) Pub. No.: US 2024/0100068 A1
(43) Pub. Date: Mar. 28, 2024

(54) METHODS COMPRISING ADMINISTRATION OF A GLUCOCORTICOID RECEPTOR AGONIST AND AN INHIBITOR OF CALCINEURIN

(71) Applicant: Chengdu Anticancer Bioscience, Ltd., Chengdu (CN)

(72) Inventors: Dun Yang, Chengdu (CN); Thaddeus Allen, Daly City, CA (US); Jing Zhang, Chengdu (CN); Shenqiu Zhang, St. Andrews (GB); Qiong Shi, Chengdu (CN); Yan Long, Chengdu (CN); Ting Zhang, Chengdu (CN); Zhengchi He, Chengdu (CN); Hongmei Li, Chengdu (CN); Chenglu Yang, Chengdu (CN)

(21) Appl. No.: 18/511,468

(22) Filed: Nov. 16, 2023

Related U.S. Application Data
(63) Continuation of application No. PCT/CN2022/094083, filed on May 20, 2022.

(51) Int. Cl. A61K 31/57; A61K 31/573; A61K 38/13; A61P 35/00; C12N 5/09; G01N 33/50

(52) U.S. Cl. CPC A61K 31/57; A61K 31/573; A61K 38/13; A61P 35/00; C12N 5/0693; G01N 33/5011; C12N 2503/02

(57) ABSTRACT

In certain aspects, disclosed herein are methods of treating cancer in a subject, comprising administration to the subject of (1) one or more glucocorticoid receptor agonists and (2) one or more inhibitors of calcineurin. In some embodiments, the administration of the one or more glucocorticoid receptor agonists and the one or more inhibitors of calcineurin results in restoration of contact inhibition, increased formation of intercellular junctions, reduced cell proliferation, and/or increased vacuolization of cells growing in multilayer zones. Included herein are methods for identification of novel compounds that restore contact inhibition in cancer cells.

### CROSS REFERENCE TO RELATED APPLICATIONS

The application claims priority to International Patent Application Number PCT/CN2021/094876, filed May 20, 2021, which is hereby incorporated by reference in its entirety.

### BACKGROUND

In normal cells, cell-cell and cell-matrix contacts mediate contact inhibition, which is the inhibition of cellular proliferation when cells reach confluence and make connections and they stop proliferating. In contrast, loss of contact inhibition of proliferation (CIP) is a hallmark of cancer cells, which tend to grow past confluence and become multilayered.

Contact inhibition of proliferation acts as a checkpoint that prevents uncontrolled cellular proliferation. The defect in the checkpoint might confer a unique vulnerability that becomes apparent only when the cells are overgrowing. However, how cancer cells evade contact inhibition remains unknown. There is no report in the literature of any chemicals that can re-establish contact inhibition in cancer cells. Therefore, compounds that can restore contact inhibition of cancer cells, and methods for identifying such compounds that can restore contact inhibition in cancer cells are needed.

### SUMMARY

In certain aspects, disclosed herein are methods of treating cancer in a subject, comprising administration to the subject of (1) one or more glucocorticoid receptor agonists and (2) one or more inhibitors of calcineurin. In certain aspects, disclosed herein are methods of reducing cancer cell proliferation, comprising administration to a subject (1) one or more glucocorticoid receptor agonists and (2) one or more inhibitors of calcineurin; wherein the cell proliferation is reduced compared to cancer cells that have not been contacted with the one or more glucocorticoid receptor agonists and the one or more inhibitors of calcineurin.

In certain aspects, disclosed herein are methods of reducing cancer cell proliferation in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin.

In certain aspects, disclosed herein are methods of inducing vacuolization of cancer cells, comprising administration to a subject (1) one or more glucocorticoid receptor agonists and (2) one or more inhibitors of calcineurin; wherein the vacuolization is characterized by an increase in the presence of vacuoles compared to cancer cells that have not been contacted with the one or more glucocorticoid receptor agonists and the one or more inhibitors of calcineurin.

In certain aspects, disclosed herein are method of inducing vacuolization of cancer cells in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin.

In certain aspects, disclosed herein are methods of inducing cell death of cancer cells, comprising administration to a subject (1) one or more glucocorticoid receptor agonists and (2) one or more inhibitors of calcineurin; wherein the number of cells undergoing cell death is increased compared to cancer cells that have not been contacted with the one or more glucocorticoid receptor agonists and the one or more inhibitors of calcineurin.

In certain aspects, disclosed herein are methods of inducing cell death of one or more cancer cells in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin.

In some embodiments, the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist. In some embodiments, the glucocorticoid receptor agonist comprises hydrocortisone. In some embodiments, the glucocorticoid receptor agonist comprises dexamethasone. In some embodiments, the calcineurin inhibitor comprises a cyclosporin. In some embodiments, the cyclosporin comprises cyclosporin A. In some embodiments, the one or more glucocorticoid receptor agonist comprises hydrocortisone and the more calcineurin inhibitor comprises cyclosporin A.

In some embodiments, the one or more glucocorticoid receptor agonists and the one or more inhibitors of calcineurin are administered to the subject or contacted with the cancer cells concurrently. In some embodiments, the one or more glucocorticoid receptor agonists and the one or more inhibitors of calcineurin are administered to the subject or contacted with the cancer cells sequentially. In some embodiments, the one or more glucocorticoid receptor agonists are administered to the subject or contacted with the cancer cells prior to the administration of or contact with the one or more inhibitors of calcineurin. In some embodiments, the one or more glucocorticoid receptor agonists are administered to the subject or contacted with the cancer cells after administration of or contact with the one or more inhibitors of calcineurin.

In some embodiments, the cancer cells overexpress MYC compared to an otherwise identical non-cancer cell. In some embodiments, the cancer cells overexpress MYC-Nick compared to an otherwise identical non-cancer cell. In some embodiments, the cancer cells express BRAF^{V600E}. In some embodiments, the cancer cells exhibit loss of TP53 expression.

In some embodiments, the cancer is metastatic. In some embodiments, the administration of or contacting with the one or more glucocorticoid receptor agonists and the one or more inhibitors of calcineurin results in a reduction in cancer proliferation, increased cancer cell death, or combinations thereof, compared to otherwise identical cancer cells not contacted with the one or more glucocorticoid receptor agonists and the one or more inhibitors of calcineurin. In some embodiments, the administration of or contacting with the one or more glucocorticoid receptor agonists and the one or more inhibitors of calcineurin results in a 2-fold or greater reduction in cancer cell proliferation as compared to otherwise identical cancer cells not contacted with the one or more glucocorticoid receptor agonists and the one or more inhibitors of calcineurin.

In some embodiments, the administration of or contacting with the one or more glucocorticoid receptor agonists and the one or more inhibitors of calcineurin results in a 2-fold or greater increase in cancer cell death as compared to otherwise identical cancer cells not contacted with the one or more glucocorticoid receptor agonists and the one or more inhibitors of calcineurin. In some embodiments, the cancer cell death is non-apoptotic cell death.

In some embodiments, the non-apoptotic cell death is characterized by an increase in the presence of vacuoles compared to cancer cells that have not been contacted with the one or more glucocorticoid receptor agonists and the one or more inhibitors of calcineurin. In some embodiments, the increase in the presence of vacuoles is at least 50 fold or more greater than the presence of vacuoles in cancer cells treated with the one or more inhibitors of calcineurin alone. In some embodiments, the increase in the presence of vacuoles is at least 5-fold or more greater than the presence of vacuoles in cancer cells treated with the one or more glucocorticoid receptor agonists alone. In some embodiments, the vacuoles have a maximum diameter of about 50 μm. In some embodiments, one or more of Calpain, Rab5, Rab11, LAMP1, MYC, MYC-Nick TFEB or ERM1 are localized to the vacuoles of the cells contacted with the one or more glucocorticoid receptor agonists and the one or more calcineurin inhibitors.

In some embodiments, the cancer cells exhibit increased intracellular junctions as compared to cancer cells that have not been contacted with the one or more glucocorticoid receptor agonists and the one or more calcineurin inhibitors.

In some embodiments, the cancer cells are grown in vitro. In some embodiments, wherein the cancer cells contacted with the one or more glucocorticoid receptor agonists and the one or more calcineurin inhibitors when grown to confluence, exhibit a greater area of cells growing in a single monolayer compared to an area of cells growing in a multi-layer, as compared to otherwise identical cancer cells grown to confluence and not treated with the one or more glucocorticoid receptor agonists and the one or more calcineurin inhibitors. In some embodiments, greater than 50% of the area of the cancer cells contacted with the one or more glucocorticoid receptor agonists and the one or more calcineurin inhibitors and grown to confluence, exhibit growth in a monolayer.

In some embodiments, the one or more glucocorticoid receptor agonists comprises hydrocortisone. In some embodiments, the one or more inhibitors of calcineurin comprises a cyclosporin. In some embodiments, the cyclosporin comprises cyclosporin A. In some embodiments, the one or more glucocorticoid receptor agonists comprises hydrocortisone and the one or more calcineurin inhibitors comprises cyclosporin A.

In certain aspects, described here are methods of screening for compounds that restore contact inhibition in a population of cancer cells, the method comprising: (i) contacting the population of cancer cells growing on a tissue culture vessel with a test compound in vitro; (ii) monitoring the viability and morphology of the cancer cells after the population of cells have grown to about 100% or more confluence to create a monolayer of cells on the bottom of the culture vessel; (iii) identifying a test compound as a compound that restores contact inhibition when, compared to a distinct population of the cancer cells that has not been contacted with the test compound; wherein the population of cancer cells contacted with the test compound exhibits: (a) a decrease in the number of cells growing as a multi-layer above the monolayer of the confluent cells, (b) an increase in cell death of cells growing as a multi-layer above the monolayer of confluent cells; (c) an increase in the number of cells with intercellular junctions, and (d) an increase in cell death of cells that have reduced intercellular junctions as compared to normal cells. In some embodiments, the population of cancer cells are contacted with the test compound when the population of cancer cells are grown to about 100% confluence or more. In some embodiments, the population of cancer cells contacted with the test compound further exhibit reduced anchorage independent growth when grown in soft agar in vitro, as compared to the population of cells that has not been contacted with the test compound. In some embodiments, the population of cancer cells contacted with the test compound further exhibits an increase in the area of monolayer growth compared to the area of multilayer growth relative to cells not contacted with the test compound. In some embodiments, the population of cancer cells contacted with the test compound further exhibits an increase in the presence of vacuoles compared to the population of the cancer cells that has not been contacted with the test compound. In some embodiments, the vacuoles have a maximum diameter of about 50 μm. In some embodiments, one or more of Calpain, Rab5, Rab11, LAMP1, MYC, NYC-Nick, TFEB or ERM1 are localized to the vacuoles.

In some embodiments, the cancer cells are a cancer cell line. In some embodiments, the cancer cells overexpress MYC compared to otherwise identical non-cancer cells. In some embodiments, the cancer cells overexpress MYC-Nick compared to otherwise identical non-cancer cells. In some embodiments, the cancer cells express BRAF^{V600E}. In some embodiments, the cancer cells have a loss of TP53. In some embodiments, the increase in cell death is non-apoptotic cell death. In some embodiments, the intercellular junctions are selected from adherens junctions and tight junctions. In some embodiments, the cells growing above the monolayer of confluent cells exhibit increased phosphorylation of Ser10 of Histone 3 compared to the cells growing as a monolayer.

In certain aspects, described herein are methods of inducing vacuolation of one or more cancer cells, comprising contacting the cancer cells with 1) one or more glucocorticoid receptor agonists and (2) one or more inhibitors of calcineurin; wherein the cancer cells, following contact with the one or more glucocorticoid receptor agonists and one or more inhibitors of calcineurin, are characterized by the presence of vacuoles; wherein the vacuoles: (i) have a maximum diameter of about 50 μm; and (ii) one or more of Calpain, Rab5, Rab11, LAMP1, MYC, MYC-Nick, TFEB or ERM1 are localized to the vacuoles. In some embodiments, the one or more inhibitors of calcineurin comprises a cyclosporin. In some embodiments, the cyclosporin comprises cyclosporin A. In some embodiments, the one or more glucocorticoid receptor agonists comprises hydrocortisone. In some embodiments, wherein the one or more glucocorticoid receptor agonists comprises dexamethasone. In some embodiments, the one or more glucocorticoid receptor agonists comprises hydrocortisone and the one or more calcineurin inhibitors comprises cyclosporin A. In some embodiments, the cancer cells overexpress MYC-Nick compared to otherwise identical non-cancer cells.

In certain aspects, described herein are methods of inducing non-apoptotic cell death of one or more cancer cells, comprising contacting the cancer cells with 1) one or more glucocorticoid receptor agonists and (2) one or more inhibitors of calcineurin; wherein the cancer cells, following contact with the one or more glucocorticoid receptor agonists and one or more inhibitors of calcineurin, are characterized by the presence of vacuoles; wherein the vacuoles: (i) have a maximum diameter of about 50 μm; (ii) one or more of Calpain, Rab5, Rab11, LAMP1, MYC, MYC-Nick, TFEB or ERM1 are localized to the vacuoles; and (iii) comprise condensed and minimized nuclei at inner periphery of vacuoles. In some embodiments, the one or more inhibitors of calcineurin comprises a cyclosporin. In some embodiments, the cyclosporin comprises cyclosporin A. In some embodiments, the one or more glucocorticoid receptor agonists comprises hydrocortisone. In some embodiments, wherein the one or more glucocorticoid receptor agonists comprises dexamethasone. In some embodiments, the one or more glucocorticoid receptor agonists comprises hydrocortisone and the one or more calcineurin inhibitors comprises cyclosporin A. In some embodiments, the cancer cells overexpress MYC-Nick compared to otherwise identical non-cancer cells.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A is a graph showing that DEX has no effect on the viability of murine liver cancer cell lines cultured under sub-confluence. The isogenic murine liver cancer cell lines T2Puro and T2MycNick were passaged and exposed to vehicle or 2 μM of DEX at a confluence of 10%. The cell viability was assayed with Trypan blue exclusion assays at day 3.

FIG. 1B is a graph showing DEX has no effect on the proliferation of murine liver cancer cell lines cultured under sub-confluence. The live cell number was calculated daily with an automatic Luna cell counter and relative cell number was shown for T2Puro.

FIG. 1C is a graph showing DEX has no effect on the proliferation of murine liver cancer cell lines cultured under sub-confluence the live cell number was calculated daily with an automatic Luna cell counter and relative cell number was shown for T2MycNick.

FIG. 2A are images (left) and a graph (right) showing HC and DEX elicits vacuolation in T2MycNick cells in a concentration-dependent manner. T2MycNick cells were passaged to a confluence of 50% and then exposed to the indicated concentrations of either HC or DEX for one week. Images were acquired with an inverted tissue culture microscope and the area occupied by vacuoles were quantified with Image J and shown in the graph (right). scale bars, 5 μm. A representative experiment is shown.

FIG. 2B are images showing maintenance and expansion of vacuoles involved the continued treatment with DEX. T2MycNick cells were exposed to 2 μM of DEX for 3 days to induce vacuolation (a) and then the cells were either continuously exposure to DEX (e and f) or transferred to a drug-free media and incubated for six days (c) before being exposed to 2 μM of DEX for another 2 days (d). Images were acquired with an inverted tissue culture microscope.

FIG. 2C are images showing high cell-density is utilized by DEX to elicit vacuolation. T2MycNick cells at either 15% (left) or 100% (right) confluence were exposed to 400 nM of DEX for 4 days before images were acquired with an inverted tissue culture microscope. Scale bar, 10 μm.

FIG. 3A is a graph (top) and images (bottom) showing treatment with Hydrocortisone reduces the final cell density of T2MycNick cells. T2MycNick cells were exposed to either vehicle or 800 nM of hydrocortisone for 7 days. The cells were either numerated with a Luna cell counter or fixed and stained with PI for nuclei to reveal monolayer zones and multilayer zones.

FIG. 3B. are images (left) and a graph (right) showing treatment with Hydrocortisone reduces the final cell density in a concentration-dependent manner. T2MycNick cells were passaged to a confluence of 50% and then exposed to the indicated concentrations of Hydrocortisone for one week before stained with PI for nuclei. The area occupied by either monolayer zones or multilayer zones was calculated by Image J and shown in the graph (right).

FIG. 4A are images (left) and a graph (right) showing monolayer zones induced by Hydrocortisone and Dexamethasone display diminished levels of proliferation. T2MycNick cells were passaged to a confluence of 50% and then exposed to vehicle or the indicated concentrations of either Hydrocortisone or Dexamethasone for one week before being processed for IF analysis with an anti-Histone H3 SerlOP antibody. Fourteen random fields in each group were used for quantification.

FIG. 4B are images showing monolayer zones induced by Hydrocortisone and Dexamethasone display diminished levels of proliferation. T2MycNick cells were passaged to a confluence of 50% and then exposed to vehicle or the indicated concentrations of either Hydrocortisone or Dexamethasone for one week before being processed for IF analysis with an anti-cyclin A1 antibody.

FIG. 5A. are images showing Hydrocortisone and Dexamethasone reduce the final cell density in a murine lung cancer cell line TA2280. The cells were exposed to vehicle or the indicated concentrations of either Hydrocortisone or Dexamethasone for 2 weeks. The cells were then fixed and stained with PI for nuclei to reveal the cell density.

FIG. 5B are graphs and images showing Hydrocortisone and Dexamethasone reduce the final cell density in a lung cancer cell line A549 and a breast cancer cell line MDA-MB-231). The cells were exposed to vehicle or the indicated concentrations of either Hydrocortisone or Dexamethasone for 2 weeks. The cells were then either numerated with a Luna cell counter or fixed and stained with PI for nuclei to reveal the cell density.

FIG. 6A. are images (left) and graphs (right) showing ectopic expression of Myc-nick but not Bc1-xL primes cells to induction of vacuolation and contact inhibition. The indicated cell isogenic cell lines were exposed for 7 days to 2 μM of DEX. The cells were then imaged under an inverted tissue culture microscope (top left) or an EVOS FL Auto microscope after staining with Acridine Orange (bottom left). The area occupied by drug-induced vacuoles was quantified with Image J and data (bottom right). At the endpoint, the cells were also numerated with a Luna cell counter and relative cell number is shown (top right).

FIG. 6B. are images (left) and a graph (right) showing ectopic expression of Myc-nick promotes cell migration. T2Puro and T2MycNick cells were passaged into 6-well plates at 80% confluence 12 hours before wounding assays. Images were acquired at 0 and 24 hours after cutting a gap in confluent culture with a pippet tip. The relative gap sizes at 24 hours compared with those at 0 hour are shown in the graph (right).

FIG. 6C. are images showing ectopic expression of Myc-nick promotes the formation of filopodia. T2Puro (a) and T2MycNick (b) cells were cultured at low density and then fixed for IF analysis of Myc (left) and the ERM proteins (right).

FIG. 7. are images of the structures and names of select agonists of glucocorticoid receptor and progesterone receptor.

FIG. 8A are images of agonists of glucocorticoid receptor but not glucocorticoid elicit vacuolation and reduce the final cell density. The T2MycNick cells were passaged into 24-well plates at 80% confluence 24 hours before treated for 7-14 days to the indicated concentrations of glucocorticoids or agonists of the glucocorticoid receptor. Cells were then imaged under an inverted tissue culture microscope.

FIG. 8B are images of agonists of glucocorticoid receptor but not glucocorticoid elicit vacuolation and reduce the final cell density. The T2MycNick cells were passaged into 24-well plates at 80% confluence 24 hours before treated for 7-14 days to the indicated concentrations of glucocorticoids or agonists of the glucocorticoid receptor. Cells were then imaged under an inverted tissue culture microscope.

FIG. 8C are images of agonists of glucocorticoid receptor but not glucocorticoid elicit vacuolation and reduce the final cell density. The T2MycNick cells were passaged into 24-well plates at 80% confluence 24 hours before treated for 7-14 days to the indicated concentrations of glucocorticoids or agonists of the glucocorticoid receptor. Cells were then imaged under an EVOS FL auto microscope after staining with PI.

FIG. 8D are images of agonists of glucocorticoid receptor but not glucocorticoid elicit vacuolation and reduce the final cell density. The T2MycNick cells were passaged into 24-well plates at 80% confluence 24 hours before treated for 7-14 days to the indicated concentrations of glucocorticoids or agonists of the glucocorticoid receptor. Cells were then imaged under an EVOS FL auto microscope after staining with PI.

FIG. 8E. are images showing the glucocorticoid receptor mediates vacuolation elicited by Dexamethasone. T2MycNick cells were treated for one week with 2 μM of DEX in the presence of absence of 5 μM of Mifepristone. Cells were then imaged under an inverted tissue culture microscope.

FIG. 9. are images (left) and graphs (right) showing both Hydrocortisone and Dexamethasone suppress the anchorage-independent growth of T2MycNick cells. The T2MycNick cells were mixed with 0.35% soft agar and transferred to 24-well microplates 24 hours before exposed to vehicle, 2 μM of DEX or 5 μM of HC. Images were acquired with an inverted tissue culture microscope at 1, 9 and 22 days after initiation of drug treatment. The sizes of spheroids were quantified with Image J (right).

FIG. 10. are images showing acidic vesicles are enriched on the periphery membrane of vacuoles and in the cells nearby a vacuole. The T2MycNick cells were treated with either vehicle or 0.6 μM of Hydrocortisone for 7 days and then exposed to Lysosenor Green DND189 for 30 min before imaged with an EVOS FL auto microscope.

FIG. 11A. are images showing a component of early endosomes Rab5 is enriched on the periphery membrane of vacuoles and in the cells nearby a vacuole. The T2MycNick cells were treated with 0.6 μM of Hydrocortisone for 7 days before fixed for IF analysis of the indicated proteins. DNA was stained with DAPI. Images were acquired with an EVOS FL auto microscope.

FIG. 11B. are images showing a component of endosomes Rab 11 is enriched on the periphery membrane of vacuoles and in the cells nearby a vacuole. The T2MycNick cells were treated with 0.6 μM of Hydrocortisone for 7 days before fixed for IF analysis of the indicated proteins. DNA was stained with DAPI. Images were acquired with an EVOS FL auto microscope.

FIG. 11C. are images showing a component of lysosomes LAMP1 is enriched on the periphery membrane of vacuoles and in the cells nearby a vacuole. The T2MycNick cells were treated with 0.6 μM of Hydrocortisone for 7 days before fixed for IF analysis of the indicated proteins. DNA was stained with DAPI. Images were acquired with an EVOS FL auto microscope.

FIG. 11D. are images showing Myc, is enriched on the periphery membrane of vacuoles and in the cells nearby a vacuole. The T2MycNick cells were treated with 0.6 μM of Hydrocortisone for 7 days before fixed for IF analysis of the indicated proteins. DNA was stained with DAPI. Images were acquired with an EVOS FL auto microscope.

FIG. 11E. are images showing Calpain is enriched on the periphery membrane of vacuoles and in the cells nearby a vacuole. The T2MycNick cells were treated with 0.6 μM of Hydrocortisone for 7 days before fixed for IF analysis of the indicated proteins. DNA was stained with DAPI. Images were acquired with an EVOS FL auto microscope.

FIG. 12. are images and a graph showing condensed mini nuclei are associated with vacuoles. The T2MycNick cells were treated with 0.6 μM of Hydrocortisone for 7 days before fixed to stain DNA with DAPI. Images were acquired with an EVOS FL auto microscope. The sizes of normal nuclei in multilayer zones and condensed mini nuclei associated with a vacuole were quantified with Image J and shown in the graph (right). Scale bar, 5 μm.

FIG. 13. Are images showing vacuolar membrane and condensed mini nuclei are highly labelled by a reactive CellTracker dye. The T2MycNick cells were treated with either vehicle or 5 μM of Hydrocortisone for 7 days before exposed to CellTracker Green CMFDA for one hour. Images were acquired with an EVOS FL auto microscope.

FIG. 14A. Are images (left) and graphs (right) showing relocation of β-catenin to the cell-cell junctions in monolayer zones. The murine liver cancer cell line T2MycNick was treated with the indicated concentration of Hydrocortisone or Dexamethasone for 7 days before fixed for IF analysis of β-catenin. DNA was stained with DAPI. Images were acquired with an EVOS FL auto microscope. The relative signal density alone the line in each subpanel was quantified with Image J.

FIG. 14B. Are images (left) and graphs (right) showing relocation of ZO-1 to the cell-cell junctions in monolayer zones. The murine liver cancer cell line T2MycNick. was treated with the indicated concentration of Hydrocortisone or Dexamethasone for 7 days before fixed for IF analysis of ZO-1. DNA was stained with DAPI. Images were acquired with an EVOS FL auto microscope. The relative signal density alone the line in each subpanel was quantified with Image J.

FIG. 14C. Are images (left) and graphs (right) showing relocation of ZO-1 to the cell-cell junctions in monolayer zones. The lung cancer cell line TA2280. was treated with the indicated concentration of Hydrocortisone or Dexamethasone for 7 days before fixed for IF analysis of ZO-1. DNA was stained with DAPI. Images were acquired with an EVOS FL auto microscope. The relative signal density alone the line in each subpanel was quantified with Image J.

FIG. 14D. Are images (left) and graphs (right) showing relocation of β-catenin to the cell-cell junctions in monolayer zones. The lung cancer cell line TA2280. was treated with the indicated concentration of Hydrocortisone or Dexamethasone for 7 days before fixed for IF analysis of β-catenin. DNA was stained with DAPI. Images were acquired with an EVOS FL auto microscope. The relative signal density alone the line in each subpanel was quantified with Image J.

FIG. 14E. Are images (left) and graphs (right) showing relocation of ZO-1 to the cell-cell junctions in monolayer zones. The lung cancer cell line TA2280. was treated with the indicated concentration of Hydrocortisone or Dexamethasone for 7 days before fixed for IF analysis of ZO-1. DNA was stained with DAPI. Images were acquired with an EVOS FL auto microscope. The relative signal density alone the line in each subpanel was quantified with Image J.

FIG. 15A are graphs showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days.

FIG. 15B is a histogram showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days. Histogram shows the percentage change in tumor volume.

FIG. 15C are images showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days. Images are of gross tumors at the endpoint.

FIG. 15D is a graph showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days. The graph shows the differences in tumor weight.

FIG. 15E are images showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days. Images are of tumor-bearing mice at the endpoint.

FIG. 15F are images showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days. Images are H & E analysis of size-matched tumors among different groups.

FIG. 15G is a graph showing hydrocortisone suppresses the growth of lung tumors. Nu/Nu mice were implanted with the murine lung cancer cell line and randomized to receive daily treatment with saline, 2.5 mg/kg of Hydrocortisone or nothing when the average tumor size reached 200 mm³. Tumor sizes were measured with a caliper once every three days. The graph shows the quantification of dead cells in size-matched tumors. More than 20 random fields were examined for each sample.

FIG. 16A. are images (left) and a graph (right) showing Cyclosporin A sensitizes cells to the induction of vacuolation elicited by Hydrocortisone. The MycNick cells were treated for 10 days with Cyclosporin A (2.5 μM) and Hydrocortisone (0.3 μM) either individually or in combination before subjected to bright-field imaging under an inverted tissue culture microscope. The quantification of the area covered by vacuoles in 16A was performed with Image J and shown in the graph (right).

FIG. 16B are images showing Cyclosporin A sensitizes cells to the induction of vacuolation elicited by Hydrocortisone. The MycNick cells were treated for 10 days with Cyclosporin A (2.5 μM) and Hydrocortisone (0.3 μM) either individually or in combination before subjected to imaging with an EVOS FL auto microscope after staining the cells with lysosensor Green DND189.

FIG. 16C are images showing Cyclosporin A sensitizes cells to the induction of vacuolation elicited by Hydrocortisone. The MycNick cells were treated for 10 days with Cyclosporin A (2.5 μM) and Hydrocortisone (0.3 μM) either individually or in combination before subjected to imaging with an EVOS FL auto microscope after staining the cells with CellTracker Green CMFDA.

FIG. 16D are images (left) and a graph (right) showing Cyclosporin A sensitizes cells to the induction of vacuolation elicited by Hydrocortisone. The MycNick cells were treated for 10 days with Cyclosporin A (2.5 μM) and Hydrocortisone (0.3 μM) either individually or in combination before subjected to imaging with an EVOS FL auto microscope after staining the cells with PI for DNA. The quantification of the area covered with either monolayer or multilayer zones was performed with Image J and shown in the graph (right).

FIG. 17A are images (left) and a graph (right) showing Ascomycin sensitizes cells to the induction of vacuolation elicited by Hydrocortisone. The MycNick cells were treated for 10 days with Ascomycin and Hydrocortisone either individually or in combination before subjected to bright-field imaging under an inverted tissue culture microscope. The quantification of the area covered by vacuoles was performed with Image J.

FIG. 17B are images showing Ascomycin sensitizes cells to the induction of vacuolation elicited by Hydrocortisone. The MycNick cells were treated for 10 days with Ascomycin and Hydrocortisone either individually or in combination before subjected to imaging with an EVOS FL auto microscope after staining the cells with Lysosensor Green DND189.

FIG. 17C are images showing Ascomycin sensitizes cells to the induction of vacuolation elicited by Hydrocortisone. The MycNick cells were treated for 10 days with Ascomycin and Hydrocortisone either individually or in combination before subjected to bright-field imaging under an inverted tissue culture microscope. or imaged with an EVOS FL auto microscope after staining the cells with PI for DNA. The quantification of the area covered by monolayer or multilayer zones was performed with Image J and shown in the graph (right).

FIG. 18A are images (left) and a graph (right) showing Pimecrolimus sensitizes cells to the induction of vacuolation elicited by Hydrocortisone. The MycNick cells were treated for 10 days with Pimecrolimus and Hydrocortisone either individually or in combination before subjected to bright-field imaging under an inverted tissue culture microscope.

FIG. 18B are images showing Pimecrolimus sensitizes cells to the induction of vacuolation elicited by Hydrocortisone. The MycNick cells were treated for 10 days with Pimecrolimus and Hydrocortisone either individually or in combination before subjected to imaging with an EVOS FL auto microscope after staining the cells with lysosensor Green DND189. (18B), or PI for DNA (18C).

FIG. 18C are images (left) and a graph (right) showing Pimecrolimus sensitizes cells to the induction of vacuolation elicited by Hydro cortisone. The MycNick cells were treated for 10 days with Pimecrolimus and Hydrocortisone either individually or in combination before subjected to imaging with an EVOS FL auto microscope after staining the cells with PI for DNA. The quantification of the area covered by monolayer or multilayer zones was performed with Image J and shown in the graph (right).

FIG. 19A. are images (left) and a graph (right) showing Tacrolimus sensitizes cells to the induction of vacuolation elicited by Hydrocortisone. The MycNick cells were treated for 10 days with Tacrolimus and Hydrocortisone either individually or in combination before subjected to bright-field imaging under an inverted tissue culture microscope.

FIG. 19B. are images showing Tacrolimus sensitizes cells to the induction of vacuolation elicited by Hydrocortisone. The MycNick cells were treated for 10 days with Tacrolimus and Hydrocortisone either individually or in combination before subjected to imaging with an EVOS FL auto microscope after staining the cells with lysosensor Green DND189.

FIG. 19C. are images (left) and a graph (right) showing Tacrolimus sensitizes cells to the induction of vacuolation elicited by Hydrocortisone. The MycNick cells were treated for 10 days with Tacrolimus and Hydrocortisone either individually or in combination before subjected to imaging with an EVOS FL auto microscope after staining the cells with PI for DNA. The quantification of the area covered by monolayer or multilayer zones was performed with Image J and shown in the graph (right).

FIG. 20A. are images (left) and a graph (right) showing Hydrocortisone in combination with a Calcineurin inhibitor is more potent than either compound alone in reducing cell density of the MM lung cancer cell line. Cells were treated for 10 days with Hydrocortisone and a Calcineurin inhibitor either individually or in combination and then stained with PI for DNA before imaged with an EVOS FL auto microscope.

FIG. 20B. are images (left) and a graph (right) showing Hydrocortisone in combination with a Calcineurin inhibitor is more potent than either compound alone in reducing cell density of the human lung cancer cell line A549. Cells were treated for 10 days with Hydrocortisone and a Calcineurin inhibitor either individually or in combination and then stained with PI for DNA before imaged with an EVOS FL auto microscope.

FIG. 20C. are images (left) and a graph (right) showing Hydrocortisone in combination with a Calcineurin inhibitor is more potent than either compound alone in reducing cell density of the liver cancer cell line T2pMig. Cells were treated for 10 days with Hydrocortisone and a Calcineurin inhibitor either individually or in combination and then stained with PI for DNA before imaged with an EVOS FL auto microscope.

FIG. 21. Is a graph showing Hydrocortisone and Cyclosporin A synergistically suppress growth of lung tumors. Nu/Nu mice were implanted with the murine lung cancer cell line MM. When the average tumor size reached 200 mm³, tumor-bearing mice were randomized to receive daily treatment with Hydrocortisone and Cyclosporin A either individually or in combination. Tumor sizes were measured with a caliper once every three days.

### DETAILED DESCRIPTION

Definitions

Terms used in the claims and specification are defined as set forth below unless otherwise specified.

The term “glucocorticoid receptor agonist” as used herein, refers to a compound that can bind the glucocorticoid receptor.

The term “inhibitor of calcineurin” as used herein, refers to a compound that can inhibit the phosphatase calcineurin.

The term “CIR compound” as used herein, refers to a compound that arrests cell cycle progression only when cells are overgrowing passing confluence. In other words, a CIR compound arrest proliferation of cells cultured in high but not low density. Alternatively, the compound might elicit death in cells that are still actively proliferating when they have reached the confluence. However, the CIR compound is generally inert to the same cells when they are dividing at sub-confluence or have stopped proliferation.

The term “vacuolization” as used herein, means the increased presence of vacuoles in a cell. In certain embodiments, the vacuoles have a maximum diameter of about 50 μm.

The term “restoring contact inhibition” or “contact inhibition restoration” or “CIR” as used herein refers to the ability of cancer cells to re-establish intercellular junctions and stop proliferating when grown to confluence in vitro or when growing in vivo.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to restore contact inhibition in a cancer cell.

The term “therapeutically effective amount” is an amount that is effective to ameliorate a symptom of a disease.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

Abbreviations used in this application include the following: CIP, which refers to contact inhibition of proliferation; CIR, which refers to contact inhibition restoration; HC, which refers to hydrocortisone; and DEX, which refers to dexamethasone.

It must be noted that, as used in the specification and the appended claims, the singular forms “a,” “an” and “the” include plural referents unless the context clearly dictates otherwise.

Briefly, and as described in more detail below, described herein are methods of treating cancer comprising administering to a subject in need thereof a sufficient amount of one or more glucocorticoid receptor agonists and one or more calcineurin inhibitors. In certain aspects, described herein are methods of restoring contact inhibition of cancer cells, reducing cancer cell proliferation, inducing vacuolization of cancer cells and/or inducing cancer cell death; comprising administering to a subject in need thereof a sufficient amount of one or more glucocorticoid receptor agonists and one or more calcineurin inhibitors. Additionally, described herein are methods of identifying compounds that restore contact inhibition, reduce cancer cell proliferation, induce vacuolization of cancer cells and/or induce cancer cell death.

Glucocorticoid Receptor Agonists

In certain aspects, described herein are methods comprising administration to the subject of one or more glucocorticoid receptor agonists. Glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide glucocorticoid receptor agonist. In some embodiments, the glucocorticoid is a selective glucocorticoid receptor agonist (SEGRAM). In some embodiments, the methods comprise administering 1, 2, 3, 4 or 5 distinct glucocorticoid receptor agonists.

Examples of natural glucocorticoid receptor agonists include, but are not limited to: Cortisol (hydrocortisone); 11-Dehydrocorticosterone (11-oxocorticosterone, 17-deoxycortisone)=21-hydroxypregn-4-ene-3,11,20-trione; 11-Deoxycorticosterone (deoxycortone, desoxycortone; 21-hydroxyprogesterone)=21-hydroxypregn-4-ene-3,20-dione; 11-Deoxycortisol (cortodoxone, cortexolone)=17α,21-dihydroxypregn-4-ene-3,20-dione; 11-Ketoprogesterone (11-oxoprogesterone; Ketogestin)=pregn-4-ene-3,11,20-trione; 11β-Hydroxypregnenolone=3β,11β-dihydroxypregn-5-en-20-one [1]; 11β-Hydroxyprogesterone (21-deoxycorticosterone)=11β-hydroxypregn-4-ene-3,20-dione; 11β,17α,21-Trihydroxypregnenolone=3β,11β,17α,21-tetrahydroxypregn-5-en-20-one [2]; 17α,21-Dihydroxypregnenolone=3β,11β,17α,21-trihydroxypregn-5-en-20-one [3]; 17α-Hydroxypregnenolone=3β,11β,17α-dihydroxypregn-5-en-20-one; 17α-Hydroxyprogesterone=17α-hydroxypregn-4-ene-3,11,20-trione; 18-Hydroxy-11-deoxycorticosterone=18,21-dihydroxypregn-4-ene-3,20-dione [4]; 18-Hydroxycorticosterone=11β,18,21-trihydroxypregn-4-ene-3,20-dione; 18-Hydroxyprogesterone=18-hydroxypregn-4-ene-3,20-dione [5]; 21-Deoxycortisol=11β,17α-dihydroxypregn-4-ene-3,20-dione; 21-Deoxycortisone=17α-hydroxypregn-4-ene-3,11,20-trione; 21-Hydroxypregnenolone (prebediolone)=3β,21-dihydroxypregn-5-en-20-one; Aldosterone=11β,21-dihydroxypregn-4-ene-3,18,20-trione; Corticosterone (17-deoxycortisol)=11β,21-dihydroxypregn-4-ene-3,20-dione; Cortisol (hydrocortisone)=11β,17α,21-trihydroxypregn-4-ene-3,20-dione; Cortisone=17α,21-dihydroxypregn-4-ene-3,11,20-trione; Pregnenolone=pregn-5-en-3β-ol-20-one; and Progesterone=pregn-4-ene-3,20-dione.

Examples of synthetic glucocorticoid receptor agonists, include but are not limited to: Progesterone-type; Flugestone (flurogestone)=9α-fluoro-11β,17α-dihydroxypregn-4-ene-3,20-dione; Fluorometholone=6α-methyl-9α-fluoro-11β,17α-dihydroxypregna-1,4-diene-3,20-dione; Medrysone (hydroxymethylprogesterone)=6α-methyl-11β-hydroxypregn-4-ene-3,20-dione; and Prebediolone acetate (21-acetoxypregnenolone)=3β,21-dihydroxypregn-5-en-20-one 21-acetate. Additional examples of synthetic glucocorticoid receptor agonists include, but are not limited to, progesterone derivative progestins such as chlormadinone acetate, cyproterone acetate, medrogestone, medroxyprogesterone acetate, megestrol acetate, and segesterone acetate which possess glucocorticoid activity that can manifest clinically at high dosages.

Examples of hydrocortisone-type glucocorticoid receptor agonists, include but are not limited to: Chloroprednisone=6α-chloro-17α,21-dihydroxypregna-1,4-diene-3,11,20-trione Cloprednol=6-chloro-11β,17α,21-trihydroxypregna-1,4,6-triene-3,20-dione; Difluprednate=6α,9α-difluoro-11β,17α,21-trihydroxypregna-1,4-diene-3,20-dione 17α-butyrate 21-acetate; Fludrocortisone=9α-fluoro-11β,17α,21-trihydroxypregn-4-ene-3,20-dione; Fluocinolone=6α,9α-difluoro-11β,16α,17α,21-tetrahydroxypregna-1,4-diene-3,20-dione; Fluperolone=9α-fluoro-11β,17α,21-trihydroxy-21-methylpregna-1,4-diene-3,20-dione; Fluprednisolone=6α-fluoro-11β,17α,21-trihydroxypregna-1,4-diene-3,20-dione; Loteprednol=11β,17α,dihydroxy-21-oxa-21-chloromethylpregna-1,4-diene-3,20-dione; Methylprednisolone=6α-methyl-11β,17α,21-trihydroxypregna-1,4-diene-3,20-dione; Prednicarbate=11β,17α,21-trihydroxypregna-1,4-diene-3,20-dione 17α-ethylcarbonate 21-propionate; Prednisolone=11β,17α,21-trihydroxypregna-1,4-diene-3,20-dione; Prednisone=17α,21-dihydroxypregna-1,4-diene-3,11,20-trione; Tixocortol=11β,17α-dihydroxy-21-sulfanylpregn-4-ene-3,20-dione; and Triamcinolone=9α-fluoro-11β,16α,17α,21-tetrahydroxypregna-1,4-diene-3,20-dione.

Examples of methasone-type glucocorticoid receptor agonists, include but are not limited to: Alclometasone=7α-chloro-11β,17α,21-trihydroxy-16α-methylpregna-1,4-diene-3,20-dione; Beclometasone=9α-chloro-11β,17α,21-trihydroxy-16β-methylpregna-1,4-diene-3,20-dione; Betamethasone=9α-fluoro-11β,17α,21-trihydroxy-16β-methylpregna-1,4-diene-3,20-dione; Clobetasol=9α-fluoro-11β,17α-dihydroxy-16β-methyl-21-chloropregna-1,4-diene-3,20-dione; Clobetasone=9α-fluoro-16β-methyl-17α-hydroxy-21-chloropregna-1,4-diene-3,11,20-trione; Clocortolone=6α-fluoro-9α-chloro-11β,21-dihydroxy-16α-methylpregna-1,4-diene-3,20-dione; Desoximetasone=9α-fluoro-11β,21-dihydroxy-16α-methylpregna-1,4-diene-3,20-dione; Dexamethasone=9α-fluoro-11β,17α,21-trihydroxy-16α-methylpregna-1,4-diene-3,20-dione; Diflorasone=6α,9α-difluoro-11β,17α,21-trihydroxy-16β-methylpregna-1,4-diene-3,20-dione; Difluocortolone=6α,9α-difluoro-11β,21-dihydroxy-16α-methylpregna-1,4-diene-3,20-dione; Fluclorolone=6α-fluoro-9α,11β-dichloro-16α,17α,21-trihydroxypregna-1,4-dien-3,20-dione; Flumetasone=6α,9α-difluoro-11β,17α,21-trihydroxy-16α-methylpregna-1,4-diene-3,20-dione; Fluocortin=6α-fluoro-11β,21-dihydroxy-16α-methylpregna-1,4-diene-3,20,21-trione; Fluocortolone=6α-fluoro-11β,21-dihydroxy-16α-methylpregna-1,4-diene-3,20-dione; Fluprednidene=9α-fluoro-11β,17α,21-trihydroxy-16-methylenepregna-1,4-diene-3,20-dione; Fluticasone=6α,9α-difluoro-11β,17α-dihydroxy-16α-methyl-21-thia-21-fluoromethylpregna-1,4-dien-3,20-dione; Fluticasone furoate=6α,9α-difluoro-11β,17α-dihydroxy-16α-methyl-21-thia-21-fluoromethylpregna-1,4-dien-3,20-dione 17α-(2-furoate); Halometasone=2-chloro-6α,9α-difluoro-11β,17α,21-trihydroxy-16α-methylpregna-1,4-diene-3,20-dione; Meprednisone=16β-methyl-17α,21-dihydroxypregna-1,4-diene-3,11,20-trione; Mometasone=9α,21-dichloro-11β,17α-dihydroxy-16α-methylpregna-1,4-diene-3,20-dione; Mometasone furoate=9α,21-dichloro-11β,17α-dihydroxy-16α-methylpregna-1,4-diene-3,20-dione 17α-(2-furoate); Paramethasone=6α-fluoro-11β,17α,21-trihydroxy-16α-methylpregna-1,4-diene-3,20-dione; Prednylidene=11β,17α,21-trihydroxy-16-methylenepregna-1,4-diene-3,20-dione; Rimexolone=11β-hydroxy-16α,17α,21-trimethylpregna-1,4-dien-3,20-dione; and Ulobetasol (halobetasol)=6α,9α-difluoro-11β,17α-dihydroxy-16β-methyl-21-chloropregna-1,4-diene-3,20-dione.

Examples of acetonide related glucocorticoid receptor agonists, include but are not limited to: Amcinonide=9α-fluoro-11β,16α,17α,21-tetrahydroxypregna-1,4-diene-3,20-dione cyclic 16α,17α-acetal with cyclopentanone, 21-acetate; Budesonide=11β,16α,17α,21-tetrahydroxypregna-1,4-diene-3,20-dione cyclic 16α,17α-acetal with butyraldehyde; Ciclesonide=11β,16α,17α,21-tetrahydroxypregna-1,4-diene-3,20-dione cyclic 16α,17α-acetal with (R)-cyclohexanecarboxaldehyde, 21-isobutyrate; Deflazacort=11β,21-dihydroxy-2′-methyl-5′H-pregna-1,4-dieno[17,16-d]oxazole-3,20-dione 21-acetate; Desonide =11β,16α,17α,21-tetrahydroxypregna-1,4-diene-3,20-dione cyclic 16α,17α-acetal with acetone; Formocortal (fluoroformylone)=3-(2-chloroethoxy)-9α-fluoro-11β,16α,17α,21-tetrahydroxy-20-oxopregna-3,5-diene-6-carboxaldehyde cyclic 16α,17α-acetal with acetone, 21-acetate; Fluclorolone acetonide (flucloronide)=6α-fluoro-9α,11β-dichloro-16α,17α,21-trihydroxypregna-1,4-dien-3,20-dione cyclic 16α,17α-acetal with acetone; Fludroxycortide (flurandrenolone, flurandrenolide)=6α-fluoro-11β,16α,17α,21-tetrahydroxypregn-4-ene-3,20-dione cyclic 16α,17α-acetal with acetone; Flunisolide=6α-fluoro-11β,16α,17α,21-tetrahydroxypregna-1,4-diene-3,20-dione cyclic 16α,17α-acetal with acetone; Fluocinolone acetonide=6α,9α-difluoro-11β,16α,17α,21-tetrahydroxypregna-1,4-diene-3,20-dione cyclic 16α,17α-acetal with acetone; Fluocinonide=6α,9α-difluoro-11β,16α,17α,21-tetrahydroxypregna-1,4-diene-3,20-dione cyclic 16α,17α-acetal with acetone, 21-acetate; Halcinonide=9α-fluoro-11β,16α,17α-trihydroxy-21-chloropregn-4-ene-3,20-dione cyclic 16α,17α-acetal with acetone; and Triamcinolone acetonide=9α-fluoro-11β,16α,17α,21-tetrahydroxypregna-1,4-diene-3,20-dione cyclic 16α,17α-acetal with acetone.

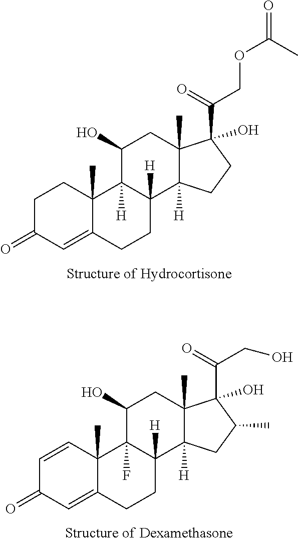

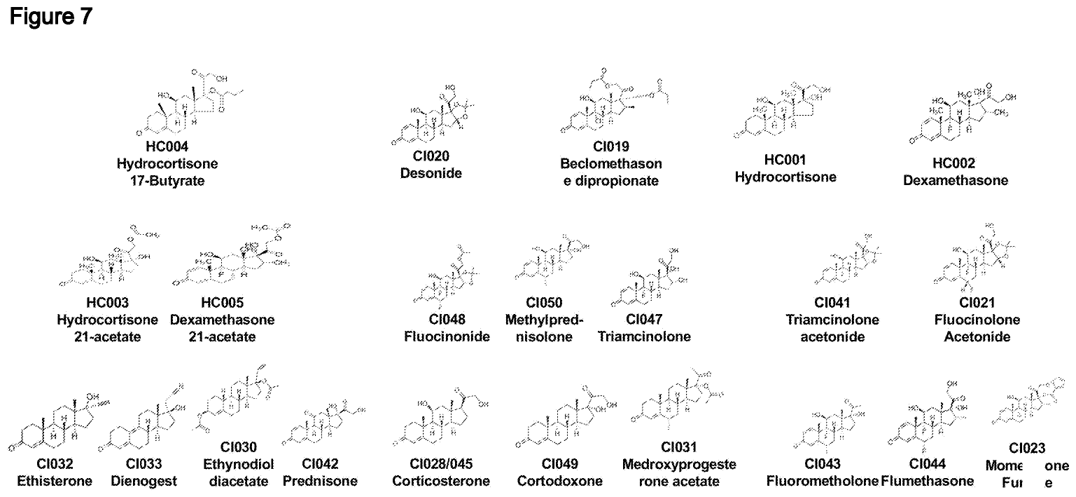

The structures of additional select glucocorticoid receptor agonists are shown in FIG. 7.

In some embodiments, the glucocorticoid receptor agonists described herein can bind to one or more of glucocorticoid receptor isoforms. Upon binding of the glucocorticoid receptor agonists to the glucocorticoid receptor, the glucocorticoid receptor can be transported to the nucleus to transcriptionally activate or transcriptionally repress expression of target genes. Upon binding of the glucocorticoid receptor agonists to the glucocorticoid receptor, the glucocorticoid receptor can modulate the physicochemical properties of membrane lipids as well as Mitogen-Activated Protein Kinase (MAPK) and other signaling cascades. Upon binding of the glucocorticoid receptor agonists to the glucocorticoid receptor, the glucocorticoid receptor can translocate to the mitochondria and regulate the gene expression in the mitochondria.

In some embodiments, the glucocorticoid receptor agonist has a potency 0.01-1000 fold the potency of hydrocortisol or cortisol. In some embodiments, the glucocorticoid receptor agonist has a potency that is 0.01-0.1 fold, 0.1-1.0-fold, 1.0-10 fold, 10-20 fold, 20-30 fold, 30-40 fold, 40-50 fold, 50-60 fold, 60-70 fold, 80-90 fold, 90-100 fold, 100-200 fold, 200-300 fold, 300-400 fold, 400-500 fold, or 500-100 fold the potency of hydrocortisol or cortisol.

In some embodiments, the glucocorticoid receptor agonist binds to the glucocorticoid receptor with an affinity that is 0.01-1000 fold the affinity of hydrocortisol or cortisol. In some embodiments, the glucocorticoid receptor agonist has an affinity to glucocorticoid receptor that is 0.01-0.1 fold, 0.1-1.0-fold, 1.0-10 fold, 10-20 fold, 20-30 fold, 30-40 fold, 40-50 fold, 50-60 fold, 60-70 fold, 80-90 fold, 90-100 fold, 100-200 fold, 200-300 fold, 300-400 fold, 400-500 fold, or 500-1000 fold the affinity of hydrocortisol or cortisol to the glucocorticoid receptor.

In some embodiments, the glucocorticoid receptor agonist promotes a glucocorticoid conformation that favors the monomer form of the glucocorticoid and inhibits or partially inhibits dimerization of the glucocorticoid receptor. In some embodiments, the glucocorticoid receptor agonist promotes a glucocorticoid conformation that favors the dimerized form of the glucocorticoid and promotes dimerization of the glucocorticoid receptor. In some embodiments, the glucocorticoid receptor agonists trigger heterodimerization between glucocorticoid receptor (GR) and mineralocorticoid receptor (MR). In some embodiments, the glucocorticoid receptor agonists is specific for the GR and activates GR/GR homodimers and not GR/MR heterodimers.

Inhibitors of Calcineurin

Calcineurin inhibitors are potent immunosuppressants that revolutionized organ transplantation since their development in the 1980s. Calcineurin inhibitors comprise three compounds: cyclosporine, tacrolimus and pimecrolimus. Tacrolimus (FK506), ascomycin (FK520) and pimecrolimus bind to their receptor macrophilin-12 (FKBP-12) and the resulting complexes inhibit the phosphatase activity of calcineurin. Likewise, cyclosporin A displays high affinity for cyclophilin, which belongs to a family of intracellular proteins called the immunophilins, and the cyclosporin A-cyclophilin complex forms a ternary complex with calcineurins, inhibiting their phosphatase activity.

In certain aspects, described herein are methods comprising administration to the subject of one or more calcineurin inhibitors. In some embodiments, the calcineurin inhibitor binds to cyclophilin. In some embodiments, the calcineurin inhibitor comprises a cyclosporine. In some embodiments, the cyclosporine comprises cyclosporine A. In some embodiments, the calcineurin inhibitor comprises voclosporin. In some embodiments, the calcineurin inhibitor binds to FK-binding protein. In some embodiments, the calcineurin inhibitor comprises Tacrolimus. In some embodiments, the calcineurin inhibitor binds macrophilin-12. In some embodiments, the calcineurin inhibitor comprises pimecrolimus.

In some embodiments, the methods comprise administering 1, 2, or 3 distinct calcineurin inhibitors.

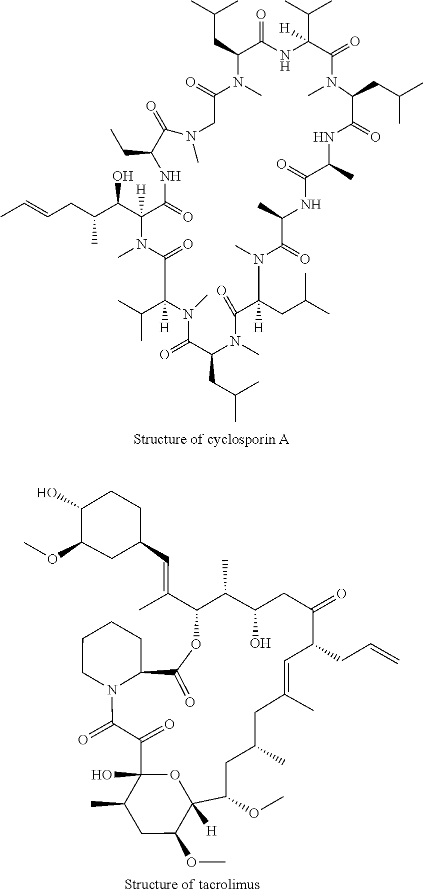

Pharmaceutical Compositions

The glucocorticoid receptor agonists and calcineurin inhibitors of the present disclosure can be formulated in one or more pharmaceutical compositions. These compositions can comprise, in addition to one or more of the glucocorticoid receptor agonists and calcineurin inhibitors, a pharmaceutically acceptable excipient, carrier, buffer, stabiliser or other materials well known to those skilled in the art. Such materials should be non-toxic and should not interfere with the efficacy of the active ingredient.

The glucocorticoid receptor agonists and calcineurin inhibitors according to the present disclosure that is to be given to an individual, administration is preferably in a “therapeutically effective amount” that is sufficient to show benefit to the individual. A “prophylactically effective amount” can also be administered, when sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

Methods of Treating Cancer

In certain aspects, disclosed herein are methods of treating cancer in a subject, comprising administration to the subject of (1) one or more glucocorticoid receptor agonists and (2) one or more inhibitors of calcineurin. In some embodiments, the administration of the one or more glucocorticoid receptor agonists and the one or more inhibitors of calcineurin results in a reduction in cancer proliferation, increased cancer cell death, or combinations thereof, compared to otherwise identical cancer cells not contacted with the one or more glucocorticoid receptor agonists and the one or more inhibitors of calcineurin.

In some embodiments, the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist.

In some embodiments, the glucocorticoid receptor agonist comprises hydrocortisone. In some embodiments, the glucocorticoid receptor agonist comprises dexamethasone. In some embodiments, the calcineurin inhibitor comprises a cyclosporin. In some embodiments, the cyclosporin comprises cyclosporin A. In some embodiments, the one or more glucocorticoid receptor agonist comprises hydrocortisone and the more calcineurin inhibitor comprises cyclosporin A.

In some embodiments, the one or more glucocorticoid receptor agonists and the one or more inhibitors of calcineurin are administered to the subject or contacted with the cancer cells concurrently. In some embodiments, the one or more glucocorticoid receptor agonists and the one or more inhibitors of calcineurin are administered to the subject or contacted with the cancer cells sequentially. In some embodiments, wherein the one or more glucocorticoid receptor agonists are administered to the subject or contacted with the cancer cells prior to the administration of or contact with the one or more inhibitors of calcineurin. In some embodiments, the one or more glucocorticoid receptor agonists are administered to the subject or contacted with the cancer cells after administration of or contact with the one or more inhibitors of calcineurin.

In some embodiments, the cancer cells overexpress MYC compared to an otherwise identical non-cancer cell. In some embodiments, the cancer cells overexpress MYC-Nick compared to an otherwise identical non-cancer cell. Myc-nick is a cytoplasmic form of Myc generated by calpain-dependent proteolysis at lysine 298 of the full-length Myc. Myc-nick inhibits apoptosis, promotes anchorage-independent growth, and renders cancer cells resistance to a variety of chemotherapeutics in part by promoting autophagy. Moreover, Myc-nick increases acetylation of α-tubulin through the recruitment of GCN5, an acetyltransferase, and promotes formation of filopodia and migration of cancer cells by induction of the actin-bundling protein fascin. In some embodiments, the cancer cells express BRAF^{V600E}. In some embodiments, the cancer cells exhibit loss of TP53 expression. Any method known in the art can be used for determining the expression of a gene in the cancer cell (e.g., immunohistochemistry and the like), and for determining the presence of a mutation of a gene can be used (e.g., sequencing methods).

In some embodiments, the administration to the subject of (1) one or more glucocorticoid receptor agonists and (2) one or more inhibitors of calcineurin results in reduced tumor volume in the subject compared to subjects not treated with the one or more glucocorticoid receptor agonists and the one or more inhibitors of calcineurin, or compared to historical controls.

In some embodiments, the cancer is lung cancer. In come embodiments, the cancer is breast cancer. In some embodiments, the cancer is metastatic.

Methods of Reducing Cancer Cell Proliferation

In certain aspects, described herein are methods of reducing cancer cell proliferation, comprising administration to the subject (1) one or more glucocorticoid receptor agonists and (2) one or more inhibitors of calcineurin; wherein the cell proliferation is reduced compared to cancer cells that have not been contacted with the one or more glucocorticoid receptor agonists and the one or more inhibitors of calcineurin. In some embodiments, the administration of or contacting with the one or more glucocorticoid receptor agonists and the one or more inhibitors of calcineurin results in a 2-fold or greater reduction in cancer cell proliferation as compared to otherwise identical cancer cells not contacted with the one or more glucocorticoid receptor agonists and the one or more inhibitors of calcineurin.

In certain aspects, described herein are methods of reducing cancer cell proliferation in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin. In some embodiments, there is increased cell proliferation in the cancer cells when contacted with the one or more glucocorticoid receptor agonists and one or more inhibitors of calcineurin when the cells are grown in sub-confluent conditions as compared to otherwise identical cancer cells that are contacted with the one or more glucocorticoid receptor agonists and one or more inhibitors of calcineurin when grown in confluent conditions of about 100% or more confluency.

In some embodiments, the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist.

In some embodiments, the glucocorticoid receptor agonist comprises hydrocortisone. In some embodiments, the glucocorticoid receptor agonist comprises dexamethasone. In some embodiments, the calcineurin inhibitor comprises a cyclosporin. In some embodiments, the cyclosporin comprises cyclosporin A. In some embodiments, the one or more glucocorticoid receptor agonist comprises hydrocortisone and the more calcineurin inhibitor comprises cyclosporin A.

In some embodiments, the one or more glucocorticoid receptor agonists and the one or more inhibitors of calcineurin are administered to the subject or contacted with the cancer cells concurrently. In some embodiments, the one or more glucocorticoid receptor agonists and the one or more inhibitors of calcineurin are administered to the subject or contacted with the cancer cells sequentially. In some embodiments, wherein the one or more glucocorticoid receptor agonists are administered to the subject or contacted with the cancer cells prior to the administration of or contact with the one or more inhibitors of calcineurin. In some embodiments, the one or more glucocorticoid receptor agonists are administered to the subject or contacted with the cancer cells after administration of or contact with the one or more inhibitors of calcineurin.

In some embodiments, cancer cells that have reduced proliferation after being administered to the subject or contacted with the one or more glucocorticoid receptor agonists and the one or more inhibitors of calcineurin also have restored localization of intercellular junction proteins to the intercellular junctions. In some embodiments, cancer cells that stopped proliferating after contact with the one or more glucocorticoid receptor agonists and the one or more inhibitors of calcineurin have re-established formation of intercellular junctions.

In some embodiments, cancer cells grown in vitro that have stopped proliferating after contact with the one or more glucocorticoid receptor agonists and the one or more inhibitors of calcineurin have re-established formation of intercellular junctions and/or are growing in a monolayer of confluent cells. Conversely, in some embodiments, the cells growing above the monolayer of confluent cells (in multilayer areas), exhibit increased cell proliferation as compared to the cells growing as a monolayer of confluent cells. In some embodiments, the cells growing above the monolayer of confluent cells (in multilayer areas), exhibit increased phosphorylation of Ser10 of Histone 3 compared to the cells growing as a monolayer of confluent cells.

In some embodiments, the cancer cells overexpress MYC compared to an otherwise identical non-cancer cell. In some embodiments, the cancer cells overexpress MYC-Nick compared to an otherwise identical non-cancer cell. In some embodiments, the cancer cells express BRAF^{V600E}. In some embodiments, the cancer cells exhibit loss of TP53 expression. In some embodiments, the cancer is lung cancer. In come embodiments, the cancer is breast cancer. In some embodiments, the cancer is metastatic.

Methods of Restoring Contact Inhibition of Cancer Cells

In certain aspects, disclosed herein are methods of restoring contact inhibition, comprising administration to the subject (1) one or more glucocorticoid receptor agonists and (2) one or more inhibitors of calcineurin; wherein the cell proliferation is reduced compared to cancer cells that have not been contacted with the one or more glucocorticoid receptor agonists and the one or more inhibitors of calcineurin. In some embodiments, the cancer cells exhibit increased intracellular junctions as compared to cancer cells that have not been contacted with the one or more glucocorticoid receptor agonists and the one or more calcineurin inhibitors.

In some embodiments, the cancer cells are grown in vitro. In some embodiments, the cancer cells contacted with the one or more glucocorticoid receptor agonists and the one or more calcineurin inhibitors when grown to confluence, exhibit a greater area of cells growing in a single monolayer compared to an area of cells growing in a multilayer, as compared to otherwise identical cancer cells grown to confluence and not treated with the one or more glucocorticoid receptor agonists and the one or more calcineurin inhibitors. In some embodiments, greater than 50% of the area of the cancer cells contacted with the one or more glucocorticoid receptor agonists and the one or more calcineurin inhibitors and grown to confluence, exhibit growth in a monolayer. In some embodiments, the cells growing above the monolayer of confluent cells exhibit increased phosphorylation of Ser10 of Histone 3 compared to the cells growing as a monolayer. In some embodiments, the cells growing above the monolayer of confluent cells exhibit increased vacuolization.

In some embodiments, the cancer cells contacted with the one or more glucocorticoid receptor agonists and the one or more calcineurin inhibitors when grown to confluence have increased intercellular junctions as compared to normal cells. Increased intercellular junctions refers to cells that exhibit increased localization of markers of one or more intercellular junctions (e.g., tight junctions and adherens junctions) compared to otherwise identical cancers cells not contacted with the one or more glucocorticoid receptor agonists and the one or more calcineurin inhibitors when grown to confluence.

In some embodiments, the population of cancer cells contacted with the one or more glucocorticoid receptor agonists and the one or more calcineurin inhibitors when grown to confluence exhibit reduced anchorage independent growth when grown in soft agar in vitro, as compared to the population of cells that has not been contacted with the test compound. In some embodiments, the population of cancer cells contacted with the test compound exhibits an increase in the area of monolayer growth compared to the area of multilayer growth relative to cells not treated with the test compound.

In some embodiments, the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist.

In some embodiments, the glucocorticoid receptor agonist comprises hydrocortisone. In some embodiments, the glucocorticoid receptor agonist comprises dexamethasone. In some embodiments, the calcineurin inhibitor comprises a cyclosporin. In some embodiments, the cyclosporin comprises cyclosporin A. In some embodiments, the one or more glucocorticoid receptor agonist comprises hydrocortisone and the more calcineurin

In some embodiments, the one or more glucocorticoid receptor agonists and the one or more inhibitors of calcineurin are administered to the subject or contacted with the cancer cells concurrently. In some embodiments, the one or more glucocorticoid receptor agonists and the one or more inhibitors of calcineurin are administered to the subject or contacted with the cancer cells sequentially. In some embodiments, wherein the one or more glucocorticoid receptor agonists are administered to the subject or contacted with the cancer cells prior to the administration of or contact with the one or more inhibitors of calcineurin. In some embodiments, the one or more glucocorticoid receptor agonists are administered to the subject or contacted with the cancer cells after administration of or contact with the one or more inhibitors of calcineurin.

In some embodiments, the cancer cells overexpress MYC compared to an otherwise identical non-cancer cell. In some embodiments, the cancer cells overexpress MYC-Nick compared to an otherwise identical non-cancer cell. In some embodiments, the cancer cells express BRAF^{V600E}. In some embodiments, the cancer cells exhibit loss of TP53 expression. In some embodiments, the cancer is lung cancer. In come embodiments, the cancer is breast cancer. In some embodiments, the cancer is metastatic.

Methods of Inducing Vacuolization

In certain aspects, described herein are methods of inducing vacuolization of cancer cells, comprising administration to the subject (1) one or more glucocorticoid receptor agonists and (2) one or more inhibitors of calcineurin; wherein the vacuolization is characterized by an increase in the presence of vacuoles compared to cancer cells that have not been contacted with the one or more glucocorticoid receptor agonists and the one or more inhibitors of calcineurin.

In certain aspects, described herein are methods of inducing vacuolization of cancer cells in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin. In some embodiments, there is decreased vacuolization in the cancer cells when contacted with the one or more glucocorticoid receptor agonists and one or more inhibitors of calcineurin when the cells are grown in sub-confluent conditions as compared to otherwise identical cancer cells that are contacted with the one or more glucocorticoid receptor agonists and one or more inhibitors of calcineurin when grown in confluent conditions of about 100% or more confluency.

In some embodiments, the increase in the presence of vacuoles is at least 50 fold or more greater than the presence of vacuoles in cancer cells treated with the one or more inhibitors of calcineurin alone. In some embodiments, the increase in the presence of vacuoles is at least 5-fold or greater than the presence of vacuoles in cancer cells treated with the one or more glucocorticoid receptor agonists alone. In some embodiments, the vacuoles have a maximum diameter of about 50 μm. In some embodiments, one or more of Calpain, Rab5, Rab11, LAMP1, MYC, MYC-Nick TFEB or ERM1 are localized to the vacuoles of the cells contacted with the one or more glucocorticoid receptor agonists and the one or more calcineurin inhibitors.

In some embodiments, the increase in the presence of vacuoles is at least 50 fold or greater than the presence of vacuoles in cancer cells treated with the one or more inhibitors of calcineurin alone. In some embodiments, the increase in the presence of vacuoles is at least 5-fold or greater than the presence of vacuoles in cancer cells treated with the one or more glucocorticoid receptor agonists alone.

In some embodiments, the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist.

In some embodiments, the glucocorticoid receptor agonist comprises hydrocortisone. In some embodiments, the glucocorticoid receptor agonist comprises dexamethasone. In some embodiments, the calcineurin inhibitor comprises a cyclosporin. In some embodiments, the cyclosporin comprises cyclosporin A. In some embodiments, the one or more glucocorticoid receptor agonist comprises hydrocortisone and the more calcineurin inhibitor comprises cyclosporin A.

In some embodiments, the one or more glucocorticoid receptor agonists and the one or more inhibitors of calcineurin are administered to the subject or contacted with the cancer cells concurrently. In some embodiments, the one or more glucocorticoid receptor agonists and the one or more inhibitors of calcineurin are administered to the subject or contacted with the cancer cells sequentially. In some embodiments, wherein the one or more glucocorticoid receptor agonists are administered to the subject or contacted with the cancer cells prior to the administration of or contact with the one or more inhibitors of calcineurin. In some embodiments, the one or more glucocorticoid receptor agonists are administered to the subject or contacted with the cancer cells after administration of or contact with the one or more inhibitors of calcineurin.

In some embodiments, the cancer cells overexpress MYC compared to an otherwise identical non-cancer cell. In some embodiments, the cancer cells overexpress MYC-Nick compared to an otherwise identical non-cancer cell. In some embodiments, the cancer cells express BRAF^{V600E}. In some embodiments, the cancer cells exhibit loss of TP53 expression. In some embodiments, the cancer is lung cancer. In come embodiments, the cancer is breast cancer. In some embodiments, the cancer is metastatic.

Methods of Inducing Non-Apoptotic Cancer Cell Death

In certain aspects, described herein are methods of inducing cell death of one or more cancer cells, comprising contacting the cancer cells with 1) one or more glucocorticoid receptor agonists and (2) one or more inhibitors of calcineurin. In some embodiments, the cancer cell death is non-apoptotic cell death.

In certain aspects, described herein are methods of inducing cell death of one or more cancer cells in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin. In some embodiments, there is decreased cell death in the cancer cells when contacted with the one or more glucocorticoid receptor agonists and one or more inhibitors of calcineurin when the cells are grown in sub-confluent conditions as compared to otherwise identical cancer cells that are contacted with the one or more glucocorticoid receptor agonists and one or more inhibitors of calcineurin when grown in confluent conditions of about 100% or more confluency.

In some embodiments, the non-apoptotic cell death is characterized by an increase in the presence of vacuoles compared to cancer cells that have not been contacted with the one or more glucocorticoid receptor agonists and the one or more inhibitors of calcineurin. In some embodiments, the administration of or contacting with the one or more glucocorticoid receptor agonists and the one or more calcineurin inhibitors results in a 2-fold or greater increase in cancer cell death as compared to otherwise identical cancer cells not contacted with the one or more glucocorticoid receptor agonists and the one or more inhibitors of calcineurin.

In some embodiments, described herein is a method of inducing non-apoptotic cell death of one or more cancer cells, comprising contacting the cancer cells with 1) one or more glucocorticoid receptor agonists and (2) one or more inhibitors of calcineurin; wherein the cancer cells, following contact with the one or more glucocorticoid receptor agonists and one or more inhibitors of calcineurin, are characterized by the presence of vacuoles; wherein the vacuoles: (i) have a maximum diameter of about 50 μm; (ii) one or more of Calpain, Rab5, Rab11, LAMP1, MYC, MYC-Nick, TFEB or ERM1 are localized to the vacuoles; and (iii) comprise condensed and minimized nuclei at inner periphery of vacuoles. In some embodiments, the cancer cells overexpress MYC-Nick compared to an otherwise identical non-cancer cell.

In some embodiments, the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist.

In some embodiments, the glucocorticoid receptor agonist comprises hydrocortisone. In some embodiments, the glucocorticoid receptor agonist comprises dexamethasone. In some embodiments, the calcineurin inhibitor comprises a cyclosporin. In some embodiments, the cyclosporin comprises cyclosporin A. In some embodiments, the one or more glucocorticoid receptor agonist comprises hydrocortisone and the more calcineurin inhibitor comprises cyclosporin A.

In some embodiments, the one or more glucocorticoid receptor agonists and the one or more inhibitors of calcineurin are administered to the subject or contacted with the cancer cells concurrently. In some embodiments, the one or more glucocorticoid receptor agonists and the one or more inhibitors of calcineurin are administered to the subject or contacted with the cancer cells sequentially. In some embodiments, wherein the one or more glucocorticoid receptor agonists are administered to the subject or contacted with the cancer cells prior to the administration of or contact with the one or more inhibitors of calcineurin. In some embodiments, the one or more glucocorticoid receptor agonists are administered to the subject or contacted with the cancer cells after administration of or contact with the one or more inhibitors of calcineurin.

In some embodiments, the cancer cells overexpress MYC compared to an otherwise identical non-cancer cell. In some embodiments, the cancer cells overexpress MYC-Nick compared to an otherwise identical non-cancer cell. In some embodiments, the cancer cells express BRAF^{V600E}. In some embodiments, the cancer cells exhibit loss of TP53 expression. In some embodiments, the cancer is lung cancer. In come embodiments, the cancer is breast cancer. In some embodiments, the cancer is metastatic.

Methods Using Predicative Biomarkers for the Therapeutic Efficacy of Glucocorticoids and/or Calcineurin Inhibitors

In some aspects, described herein are methods of predicting therapeutic efficacy of glucocorticoids and/or calcineurin inhibitors for treatment of cancer. In certain embodiments, Myc-Nick is a predictive biomarker for the therapeutic efficacy of at least one glucocorticoid(s) and/or at least one calcineurin inhibitor(s). In some embodiments, the methods described herein comprise determining Myc-Nick expression in cancer cells obtained from a subject. In some embodiments, the expression or amount of expression of Myc-Nick informs the decision to whether to treat a subject harboring cancer cells with at least one glucocorticoid(s) and/or at least one calcineurin inhibitor(s). The expression of Myc-Nick can be determined by any method known in the art for detection of protein, and/or nucleic acid sequences encoding Myc-Nick.

In some embodiments, high or elevated calpain activity greater than appropriate controls predicts therapeutic efficacy of glucocorticoids and/or calcineurin inhibitors for treatment of cancer. In certain embodiments, the appropriate control for the determination of elevated calpain activity is non-cancer tissue from the same individual. In certain embodiments, high or elevated calpain activity is a predictive biomarker for the therapeutic efficacy of at least one glucocorticoid(s) and/or at least one calcineurin inhibitor(s). In certain embodiments, both expression of Myc-Nick and elevated calpain activity is a predictive biomarker for the therapeutic efficacy of at least one glucocorticoid(s) and/or at least one calcineurin inhibitor(s). In some embodiments, the methods described herein comprise determining calpain activity in cancer cells obtained from a subject. In some embodiments, high or elevated calpain activity informs the decision to treat a subject harboring cancer cells with at least one glucocorticoid(s) and/or at least one calcineurin inhibitor(s). Calpain activity can be determined by any method known in the art, including but not limited to, fluorometric assays.

Methods of Screening Compounds that Restore Contact Inhibition

In certain aspects, described herein are methods of identifying compounds that restore contact inhibition of cancer cells. In some embodiments, described herein is a method of screening for compounds that restore contact inhibition in a population of cancer cells, the method comprising: (i) contacting the population of cancer cells growing on a tissue culture vessel with a test compound in vitro; (ii) monitoring the viability and morphology of the cancer cells after the population of cells have grown to about 100% confluence to create a monolayer of cells on the bottom of the culture vessel; (iii) identifying a test compound as a compound that restores contact inhibition when, compared to a distinct population of the cancer cells that has not been contacted with the test compound; wherein the population of cancer cells contacted with the test compound exhibits: (a) a decrease in the number of cells growing as a multilayer above the monolayer of the confluent cells, (b) an increase in cell death of cells growing as a multilayer above the monolayer of confluent cells; (c) an increase in the number of cells with intercellular junctions, and (d) an increase in cell death of cells that have reduced intercellular junctions as compared to normal cells. In some embodiments, the population of cancer cells are contacted with the test compound when the population of cancer cells are grown to about 100% confluence or more. In some embodiments, the identifying the test compound as a compound that restores contact inhibition further comprises determining a reduction in cell death and/or cell proliferation in cells growing in the monolayer as compared to cells growing in the multilayer in the population of cancer cells contacted with the test compound compared to the distinct population of cancer cells that have not been contacted with the test compound. In some embodiments, the identifying the test compound as a compound that restores contact inhibition further comprises determining increased cell death, reduced vacuolization, and increased cell proliferation in the population of cancer cells contacted with the test compound when the population is grown in sub-confluent conditions as compared to a distinct population of cancer cells contacted with the test compound when the population is grown in confluent conditions of about 100% or more confluency.

Reduced intercellular junctions refers to cells that exhibit reduced localization of markers of one or more intercellular junctions (e.g., tight junctions and adherens junctions) compared to otherwise identical cells not contacted with the test compound. In some embodiments, the intercellular junctions are selected from adherens junctions and tight junctions. In some embodiments, the increase in cell death is non-apoptotic cell death. In some embodiments, the cells growing above the monolayer of confluent cells exhibit increased phosphorylation of Ser10 of Histone 3 compared to the cells growing as a monolayer. In some embodiments, the population of cancer cells contacted with the test compound exhibits an increase in the area of monolayer growth compared to the area of multilayer growth relative to cells not contacted with the test compound.

In some embodiments, the cells are grown on an extracellular matrix material (e.g., collagen). In some embodiments, a test compound is identified as a compound that restores contact inhibition if cells contacted with the compound exhibit an increase in intracellular junction formation and maintenance compared to otherwise identical cells not contacted with the test compound, as determined by staining the cells for one or more components of intercellular junctions (e.g., tight junctions and adherens junctions) and visualization by microscopy. In some embodiments, a test compound is identified as a compound that restores contact inhibition if cells contacted with the compound exhibit a decrease in the area of multilayer zones and an increase in area of monolayer zones compared to otherwise identical cells that have not been contacted with the test compound.

In some embodiments, the population of cancer cells contacted with the test compound further exhibits an increase in the presence of vacuoles compared to the population of the cancer cells that has not been contacted with the test compound. In some embodiments, the vacuoles have a maximum diameter of about 50 μm. In some embodiments, one or more of Calpain, Rab5, Rab11, LAMP1, MYC, NYC-Nick, TFEB or ERM1 are localized to the vacuoles. In some embodiments, a test compound is identified as a compound that restores contact inhibition if cells contacted with the compound exhibit an increase in vacuolization of cells in multilayer compared to otherwise identical cells in multilayer zones that have not been contacted with the test compound. In some embodiments, the vacuolization is determined by using a dye to identify acidic subcellular compartments. In some embodiments, the cells are stained for markers of endosomes and/or lysosomes to determine vacuolization.

The anchorage-independent growth of cells is one of the hallmarks of cancer cells. Normal epithelial cells are supported by basement membranes that provide survival and proliferative signals while undergo a type of apoptosis called anoikis when lose their attachment to the extracellular matrix. Cancer cells, in contrast, evade attachment-induced apoptosis, leading to uncontrolled proliferation and metastasis. Measuring the ability of cells to grow in soft agar has been popularly believed as the gold standard assay for cellular transformation in vitro. In the soft agar assay, cells grow from single cells to cell colonies in a semi-solid agar solution that keeps them away from the solid surface and allows growth in an anchorage-independent way. The soft agar colony formation assay allows testing of the therapeutic efficacy of compounds against anchorage-independent 3D growth of cancer cells in vitro. In some embodiments, the population of cancer cells contacted with the test compound exhibits reduced anchorage independent growth when grown in soft agar in vitro, as compared to the population of cells that has not been contacted with the test compound.

In some embodiments, the test compound causes a reduction in tumor volume when administered to a subject compared to subjects with cancer that have not been administered the test compound. In some embodiments the test compound reduces the metastasis of cancer cells when administered to a subject compared to subject with cancer that have not been administered the test compound.

In some embodiments, the cancer cells are a cancer cell line. In some embodiments, the cancer cell line is a mammalian cancer cell line (e.g., a murine cancer cell line). In some embodiments, the cancer cell line is a human cancer cell line. In some embodiments, the cancer cells overexpress MYC compared to otherwise identical non cancer cells. In some embodiments, the cancer cells overexpress MYC-Nick compared to otherwise identical non-cancer cells. In some embodiments, the cancer cells express BRAF^{V600E}. In some embodiments, the cancer cells have a loss of TP53. In some embodiments, the cancer cell line is a lung cancer cell line. In some embodiments, the cancer cell line is a breast cancer cell line.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

Methods

### Cell Culture

The human lung cancer cell line A549 was obtained from ATCC. The murine liver cancer cell line T2pMig was derived from a mouse liver cancer model initiated by a transgene of MYC. The MycNick cell line was generated by stably expressing MycNick, the N-terminal fragment of Myc in the T2pMig cell line. The murine lung cancer cell line MM was generated from a mouse model of lung cancer collaboratively initiated by BRAF^{V600E} and homozygous loss of the tumor suppressor TP53. All cell lines were cultured in DMEM (Gibco, Cleveland, TN, USA) supplemented with 5% fetal bovine serum (Gibco), penicillin (100 U/mL)-streptomycin (100 μg/mL) (Gibco, Cat. No.15140-122), 2 mM L-glutamine (Gibco, 200 mM solution, Cat. No. 25030081), and 1 mM sodium pyruvate (Gibco, 100 mM solution, Cat. No. 11360070) at 37° C. in a humidified incubator that was maintained at 5% CO₂.

### Chemicals

The hydrocortisone analogs and four immunosuppressives were obtained commercially and were prepared at a stock concentration of 20 mM in DMSO. The compounds purchased from Aladdin were Hydrocortisone (CAS: 50-23-7), Dexamethasone (CAS: 50-02-2), Fluocinolone Acetonide (67-73-2), Fluticasone propionate (CAS: 80474-14-2), Mometasone Furoate (CAS: 83919-23-7), Dexamethasone 21-phosphate disodium salt (CAS: 2392-39-4), Triamcinolone acetonide (CAS: 76-25-5), Fluorometholone (CAS: 426-13-1), Flumethasone (CAS: 2135-17-3), Beclomethasone dipropionate (CAS: 5534-09-8), Desonide (CAS: 638-94-8), Corticosterone (CAS: 50-22-6), Fluocinonide (CAS: 356-12-7), Methylprednisolone (CAS: 83-43-2), Medroxyprogesterone acetate (CAS: 71-58-9), Ethynodiol diacetate (CAS: 297-76-7), Ethisterone (CAS: 434-03-7), Dienogest (CAS: 65928-58-7), Prednisone (CAS: 53-03-2), Hydrocortisone 17-Butyrate (CAS: 13609-67-1), Hydrocortisone 21-acetate (CAS: 50-03-3), Dexamethasone 21-acetate (CAS: 1177-87-3), Cyclosporin A (CAS: 59865-13-3), Ascomycin (CAS: 104987-12-4), Pimecrolimus (CAS: 137071-32-0), and Tacrolimus (CAS: 104987-11-3). Triamcinolone (CAS: 124-94-7) and Cortodoxone (CAS: 152-58-9) were purchased from Selleck Chemicals. Mifepristone (RU486) (Cat. # m8046-100MG) was obtained from Sigma.

### Immunofluorescent Microscopy

Cells were cultured on collagen-coated coverslips in a 6-well plate and exposed to chemicals including hydrocortisone, its analogs, calcineurin inhibitors or a combination between hydrocortisone and a calcineurin inhibitor. Cells were transferred every three days to fresh media supplemented with the same drug or drug recombination. After the treatment lasted for 10-16 days, cells were fixed with either 4% paraformaldehyde or 4% paraformaldehyde followed with methanol treatment, and then permeabilized with 0.1% Triton X-100. After blocking with 5% BSA, cells were incubated with a primary antibody for 2 hours at room temperature.

The following are primary antibodies used in this study. Rabbit antibodies for β-Catenin (6B3) (Cat. #9582s), H3Ser10P (Cat. #9701L), cleaved caspase 3 (Asp175) (Cat. #9661L), Rab5 (C8B1) (Cat.#29655) , Rab7 (Cat. #9367P), Rab11 (Cat. #5589P), and LAMP1 (Cat. #9091P) were from Cell Signaling Technology. Mouse mAb for Calnexin (Cat.# ab2798-100), Rabbit pAb for Lamp1 (Cat.# ab24170-100), Rabbit mAb to Calpain S1 (EPR3324) (Cat.# ab92333) and Rabbit mAb for MYC (Y69) (Cat.# ab30072) were from Abcam. Rabbit anti-TFEB antibody (Cat.#A303-673A) was from Bethyl. Rabbit antibody for cyclin A (H-432) (Cat. #sc-751) was from Santa Cruz Biotechnology. Mouse mAb for ZO-1 (Cat. # 66452-1-1g) was from Proteintech. The anti-Histone H3 (Ser10 P) antibody was used after a 1000-fold dilution and the rest of the antibodies were used at a dilution of 1:100. Primary antibodies were detected with Rhodamine (TRITC)-conjugated AffiniPure Donkey Anti-Rabbit IgG (H+L) (Cat. # 711-025-152), Fluorescein(FITC)-conjugated Affinipure Goat Anti-Rabbit IgG (H+L) (Cat.#111-095-003), Alexa Fluor488 Affinipure Goat Anti-Mouse IgG (H+L) (Cat. #115-545-003 from Jackson ImmunoResearch at 1:500 dilution. After immunostaining, cells were mounted on microscope slides with 4′,6′-diamidino-2-phenylindole (DAPI)-containing Vectashield mounting solution (Vector Laboratories, Cat. # H1500). For fluorescence detection, an EVOS FL Auto microscope (Thermo Fisher) was used.

### Imaging Intracellular Acidic Organelles

For determination of acidic subcellular compartments, cells were cultured on cover slips and exposed for 40 min at 37° C. with 1 nM of LysoSensor green DND189 (Thermo Fisher, Cat. #L7535), a dye that is routinely used to measure the pH of acidic organelles such as lysosomes and becomes more fluorescent in acidic environments. Alternatively, cells were treated with 1 μM of Acridine Orange (AO) (Invitrogen, Cat. #A3568). Although quite cell permeant in the neutral form, once protonated, AO dye tends to become trapped on the low pH side of the membrane barrier leading to its accumulation in acidic organelle structures, such as lysosomes. The effectiveness of this concentration process is sufficient to create intra-lysosomal concentrations leading to precipitation of the AO dye into aggregated granules. These oligomeric structures exhibit a red shift (640 nm) compared to the monomeric AO that emits at 525 nm. Lysosomes will appear yellowish green by illuminating cells with a blue light (488 nm) excitation filter and a green light (540-550 nm) emission/barrier filter. Alternatively, lysosomes will appear red when using an excitation filter of 550 nm (540-560 nm) and a long pass >610 nm emission/barrier filter. After fixation in 4% of paraformaldehyde, dye-stained cells were mounted on microscope slides with DAPI-containing Vectashield. An EVOS FL Auto microscope (Thermo Fisher) was used to detect fluorescence.

Testing Compounds for their Ability of Restoring Contact Inhibition

Exponentially growing MycNick cells were passaged into 24-well plates at a final confluence of 90% and were allowed to attach for 48 hours before being exposed to a cortisol analog in the presence or absence of a calcineurin inhibitor. After the initiation of treatment, the cells were allowed to grow beyond confluence for 7 days before being subjected to morphological analysis and cell density. An inverted tissue culture microscope (Leica) was used to document vacuoles in live cells. Cell density was evaluated under an EVOS FL Auto microscope (Thermo Fisher) after fixation of the treated cells with 4% paraformaldehyde (PFA) in the presence of detergent 0.5% Triton X-100 and subsequently staining of DNA with 5 μg/ml of Propidium Iodide (PI) in the presence of 5 μg/ml of Ribonuclease A from bovine pancreas (RNase A) (Sigma, Cat. # R6513-50MG).

The threshold concentration that was used to elicit no less than 50% of reduction in the cell density was determined. So was the minimal effective concentration that could induce vacuolation to an extent that covered no less than 10% of a random field documented under a bright field microscope. Quantification of the area with vacuolation, multilayer zones or multilayer zones was performed with the Image J software. More than 10 random fields were chosen for quantification for each data point in each of two independent experiments. The average of these data in was presented in the figures.

### Soft Agar Colony Formation Assay

The soft agar colony formation assay was performed in 6-well plates with two layers of agar. For the first, 0.75% agar in DMEM medium was melted in a microwave oven and poured to form a bottom layer. Once solidified, 10-100K cells in 1 ml of DMEM containing 0.35% agar was added to form the top layer, which was later covered with 0.5 ml of DMEM. Cell culture medium was changed once every two days until colonies were ready to photograph. The antitumor activity of Hydrocortisone and Dexamethasone was tested in soft agar assays. Both compounds effectively suppressed the anchorage-independent growth of MycNick cells despite they have no effect on proliferation when the cells are dividing in sub-confluence in 2D culture. The suppression of growth of the cell line in 3D culture is consistent with the suppressive impact of these compounds on cell density when cells were allowed to grow beyond confluence.

### MTT Assay for Cellular Proliferation and Determination of EC₅₀

The MTT assay was performed to measure cellular metabolic activity as a proxy for cell viability and involves the conversion of the water-soluble yellow dye MTT [3-(4,5-dimethylthiazol-2-yl)-2,5-diphenyltetrazolium bromide] into an insoluble purple formazan by the action of mitochondrial reductase. Formazan is then solubilized and its concentration is determined by measuring the optical density (OD) value at a wavelength of 570 nm. The value is in proportional to the number of live cells with excellent linearity up to˜10⁶ cells per well. The MTT assay was used to determine the EC₅₀ value, the concentration of a compound that leads to 50% inhibition of cellular proliferation. Briefly, cells were split when growing to the mid-Log phase. Cells in 100 μL of culture medium were seeded into each well of 96-well microplates and cultivated for 15-24 hours to reach a confluence of 20-30% and were then exposed to drugs at concentrations ranging from 1 nM to 5 μM. At the endpoint, 20 μL of a MTT stock solution in DMSO (5 mg/mL) was added to each well that contains 100 μL of DMEM. The microplates were left in the cell culture incubator for 3-4 h before subjected to solubilization and determination of formazan at A570 in a microplate reader (BioTek ELX808iu). It was found that neither Hydrocortisone nor Cyclosporin A affected the proliferation of MycNick cells in this assay.

### Xenograft Assays

Xenografts were initiated in immunocompromised (Nu/Nu) mice with the murine lung adenocarcinoma cell line MM and liver cancer cell line MycNick. Five million cells were injected subcutaneously into each mouse and treatment was initiated when the average tumor volumes reached 150 mm³ (n=5/groups). Tumor-bearing mice were randomized into different groups to receive either vehicle or indicated compounds. The compounds were administered once a day. Hyrocortisone was administered by injection subcutaneously near the tumors whereas cyclosporine A was given through oral gavage. For these experiments, clinically used Hyrocortisone injections (5 mg/ml, H20023069) were obtained from China National Pharmaceutical Group Co., Ltd. Cyclosporin A was formulated in Sesame oil at 2 mg/ml. 100 ul of drug solution was administered with each dose. Tumor volumes were determined once every three days and are calculated from digital caliper raw data by using the formula: Volume (mm3)=(L×W²)/2. The value W (Width) is the smaller of two perpendicular tumor axes and the value L (Length) is the larger of two perpendicular axes. Mean tumor volume growth curves and means are calculated for each treatment group.

### Assessing Therapeutic Synergism Between Two Drugs

The combination index (CI) was used to evaluate therapeutic synergism between two drugs. CI=(1−TGIab)/(1−TGIa)(1−TGIb) a,b: represents two different drugs. TG1ab: Tumor growth inhibition when both drugs a and b are used in combination. TGIa: Tumor growth inhibition when drug a is administered. TGIb: Tumor growth inhibition when drug b is administered. 0.9≤CI≤1.1: superposition effect; 0.8≤CI<0.9: low degree of synergy; 0.6≤CI<0.8: Moderate synergy; 0.4≤CI<0.6: highly synergistic effect; CI<0.4: strong synergetic effect.

### Statistical Analysis

Two or multiple group comparisons were conducted by using Graphpad Prism 7.0. Analysis of variance (ANOVA) provides a statistical test of whether two or more group means are equal. Tukey or Dunnett test are then carried out if ANOVA rejects the null hypothesis that two or more-group means are equal. Tukey's test compares the means of every treatment to the means of every other treatment. Dunnett's test compares the means of every treatment to a single control.

### Example 1

### Restoration of Contact Inhibition by Hydrocortisone and Dexamethason in Cells Expressing Myc-Nick

Exponentially growing murine cells expressing Myc-Nick (T2MycNick cells) were passaged into 24-well plates at a final confluence of 20% and were allowed to attach overnight before being exposed to a test compound at a concentration of 10 μM. T2MycNick cells were generated by expressing the Myc-nick, the N-terminal of the Myc protein, in the T2 cell line, which was, in turn, derived from a MYC-driven mouse liver cancer (Shachaf et al., Nature 2004 Oct. 28; 431(7012):1112-7). The cells were subjected to daily analysis of viability and morphology under an inverted tissue culture microscope or GE InCell Analyzer 2000. Cell density was documented after staining of DNA with PI 7-10 days after the initiation of treatment.

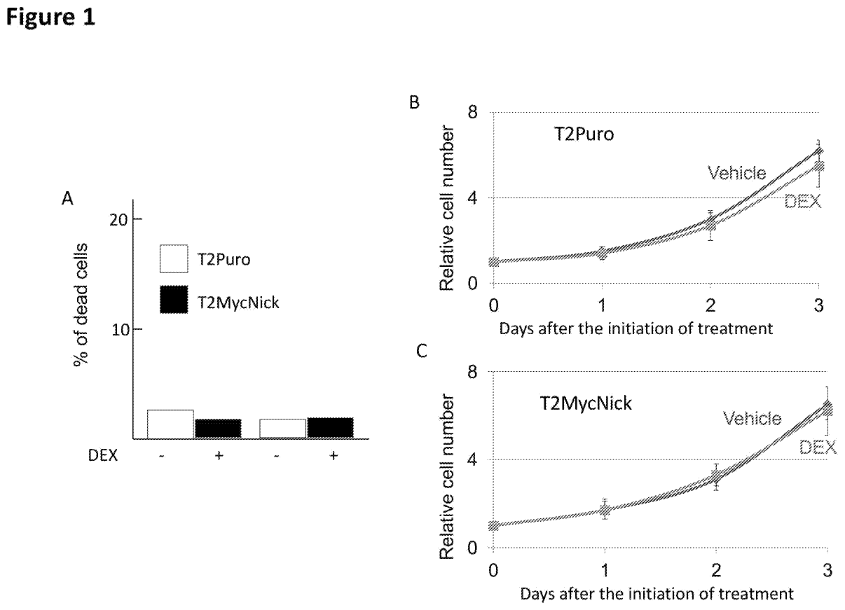

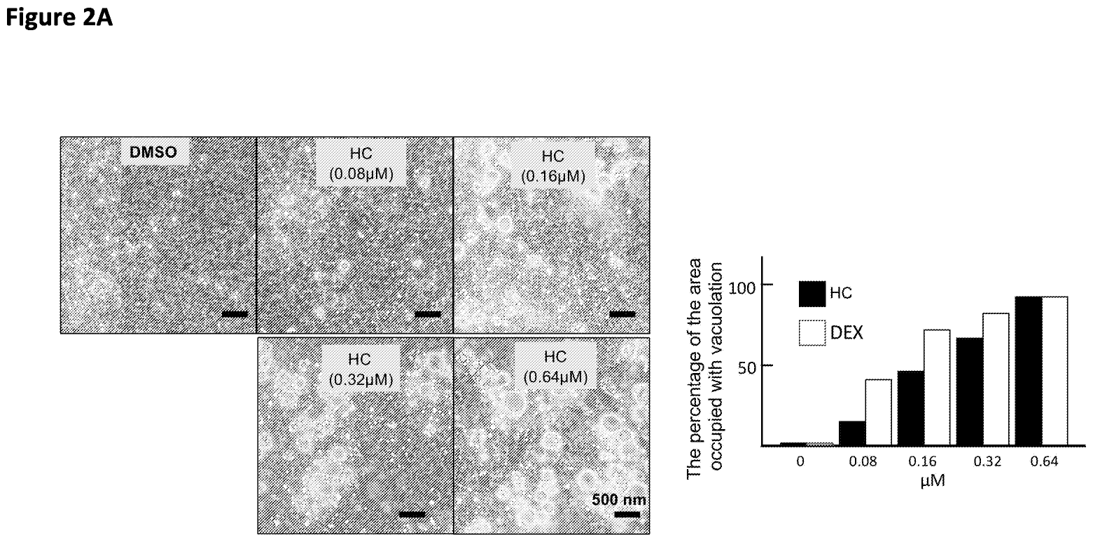

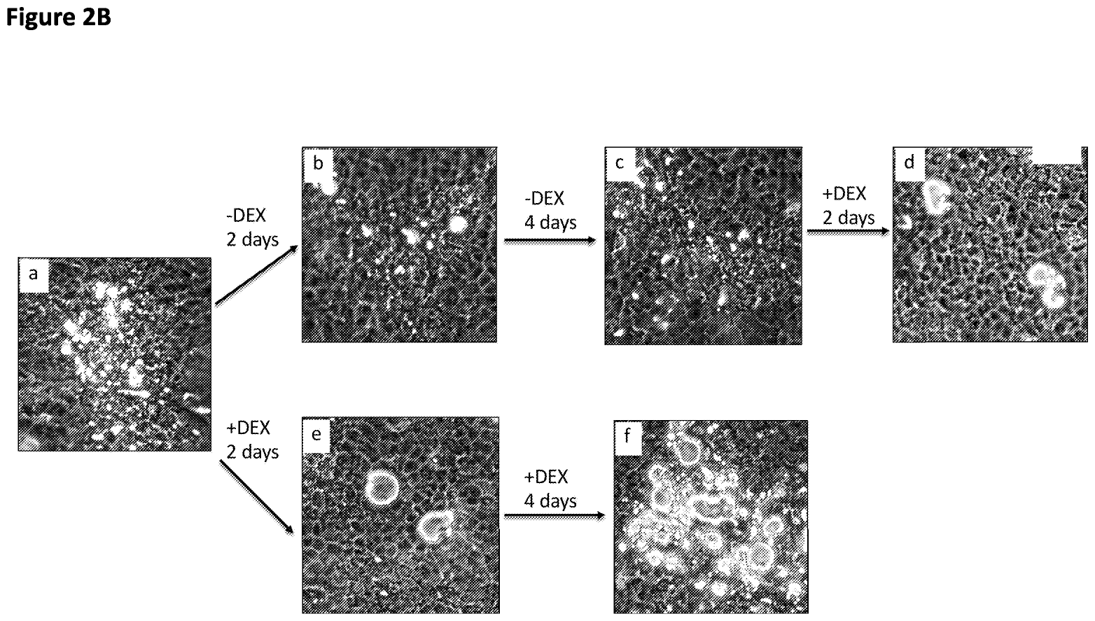

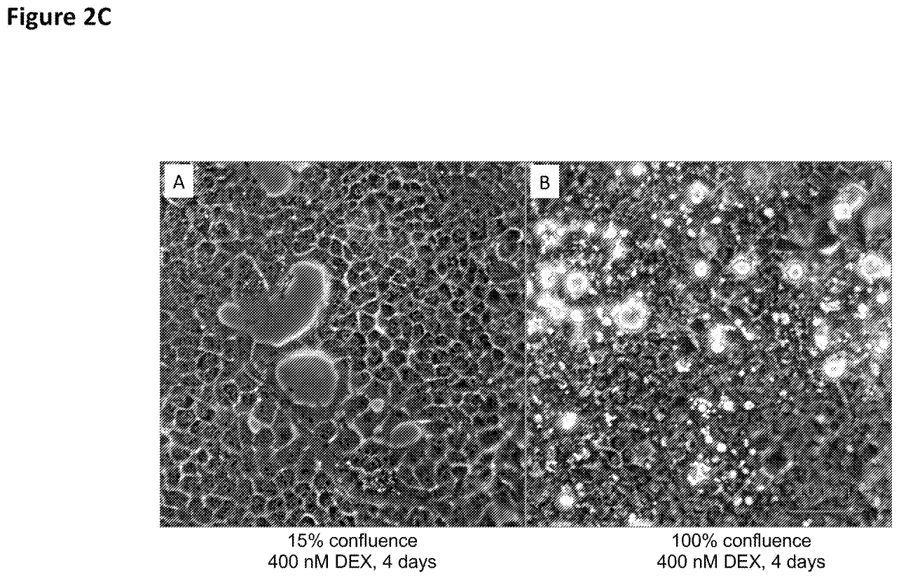

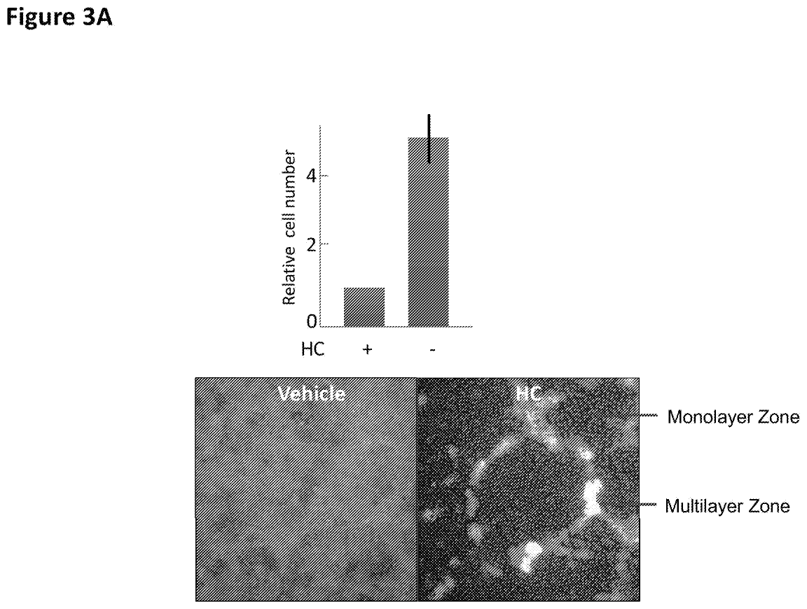

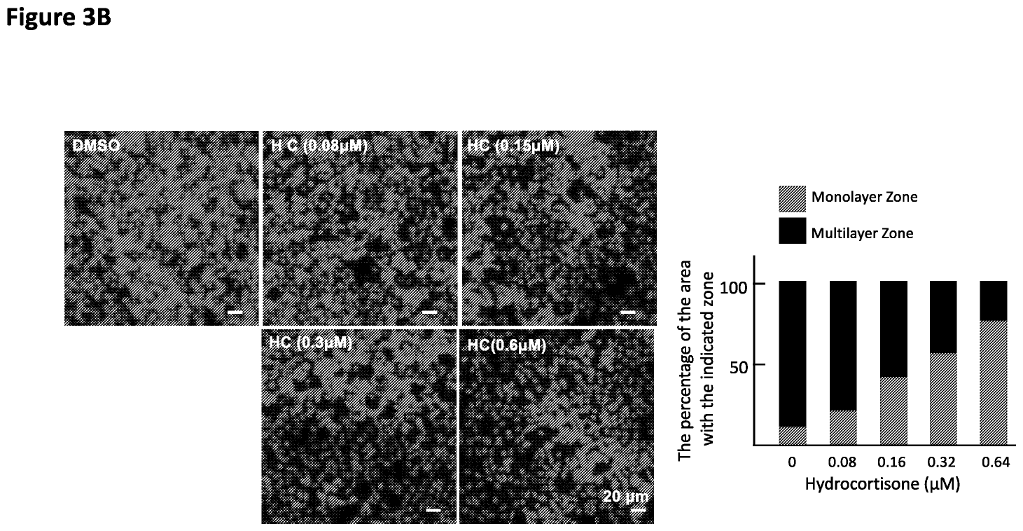

Hydrocortisone (HC) and dexamethasone (DEX) had no detectable effect on proliferation and viability when T2MycNick cells were grown at sub-confluence (FIG. 1). However, both compounds elicited marked changes in cells that overgrew beyond confluence. Specifically, they triggered the appearance of large vacuoles with diameters of about 20 μm. More extensive vacuolation occurred at higher concentrations (FIG. 2A). Maintenance and expansion of vacuoles utilized continued exposure to DEX (FIG. 2B). Under the same duration of treatment, these two compounds did not elicit such vacuolation when cells were cultured in sub-confluence or proliferatively arrested by deprivation of serum (FIG. 2C). This contrast indicates that uncontrolled proliferation of high-density cells might be useful for HC/DEX to elicit vacuolation. Furthermore, HC and DEX promoted the transformation of overgrown cells in a multilayer into localized monolayers of confluent cells (FIG. 3A). The area of monolayer zones increased steadily at the expense of multilayer zones in a drug concentration-dependent manner (FIG. 3B).

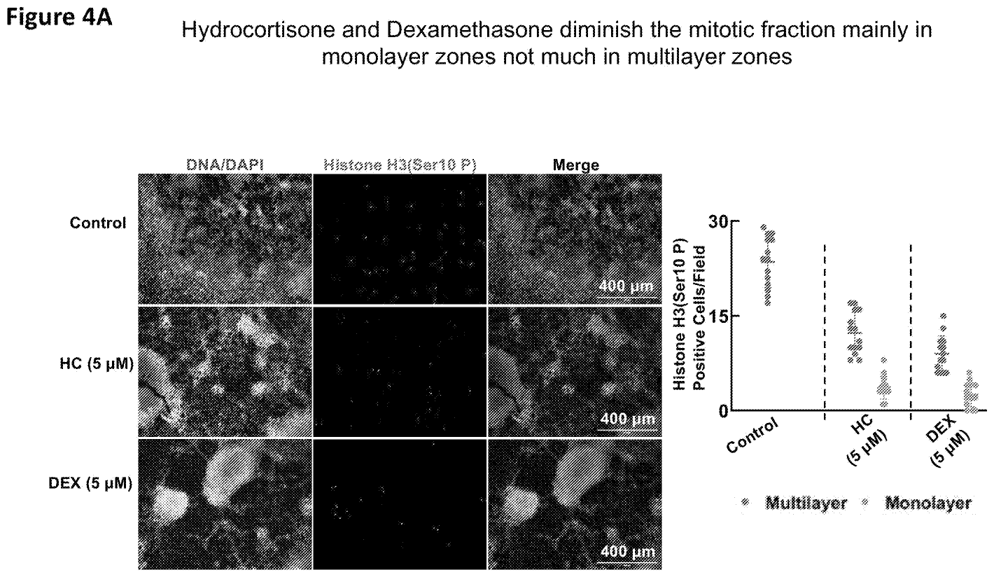

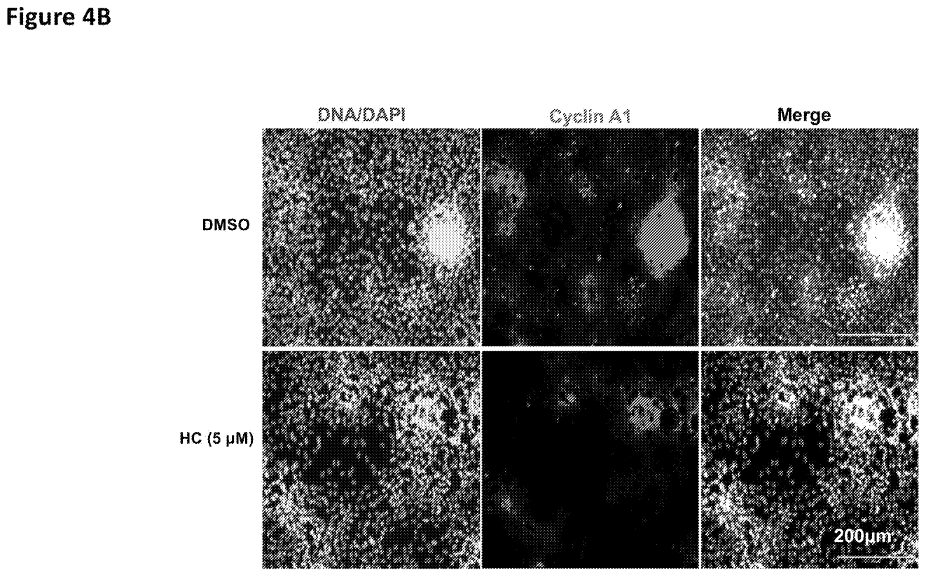

T2MycNick cells in the monolayer invariably ceased proliferation, whereas those in multilayer zones were still actively dividing, as shown by the phosphorylation of Ser10 at Histone H3 (FIG. 4A). Monolayer zones but not multilayer zones also lacked cells positive for cyclin A, which promotes the entrance into S phase from G1 in the cell cycle (FIG. 4B). Collectively, these data indicate that cells in the monolayer zones were likely arrested in the G1 phase. However, cells in multilayer zones were still proliferating at levels slower than that seen in the control group. Proliferation of T2MycNick cells under regular passage to maintain sub-confluence was not affected by chronic treatment with either HC or DEX (FIG. 1). The suppressive effect by treatment with the combination of HC and DEX on proliferation of only high-density cells implies that cell-cell contact was utilized for HC and DEX to arrest the cell cycle progression. In other words, HC and DEX restored contact inhibition, rather than inhibited cellular proliferation in general. This finding is the first documentation that a chemical compound can restore contact inhibition in tumor cells and induce large vacuoles, as noted above.

In the monolayer areas, HC and Dex did not elicit the vacuoles seen in the multilayer. HC and Dex also did not cause cell death in monolayer zones since dead cells were not detected by either the Trypan blue exclusion assay or TUNEL apoptosis assay. The cells in monolayer zones could readily resume proliferation upon passaging into low-density culture, indicating the proliferative arrest was likely transient and reversible. At the concentration of 5 μM used in this study, HC did not affect cellular proliferation and viability when these cell lines were grown in sub-confluence. A specific alteration induced by high cell-density was useful for HC and DEX to arrest the cell cycle progression. Thus, HC and DEX inhibited proliferation selectively.

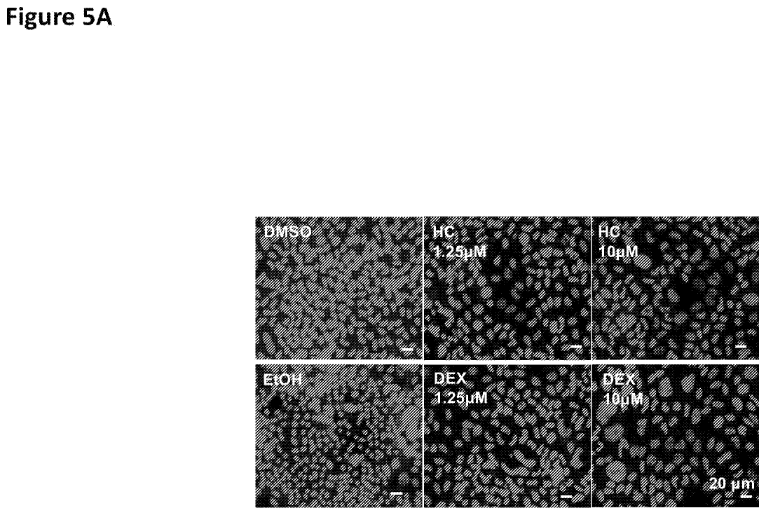

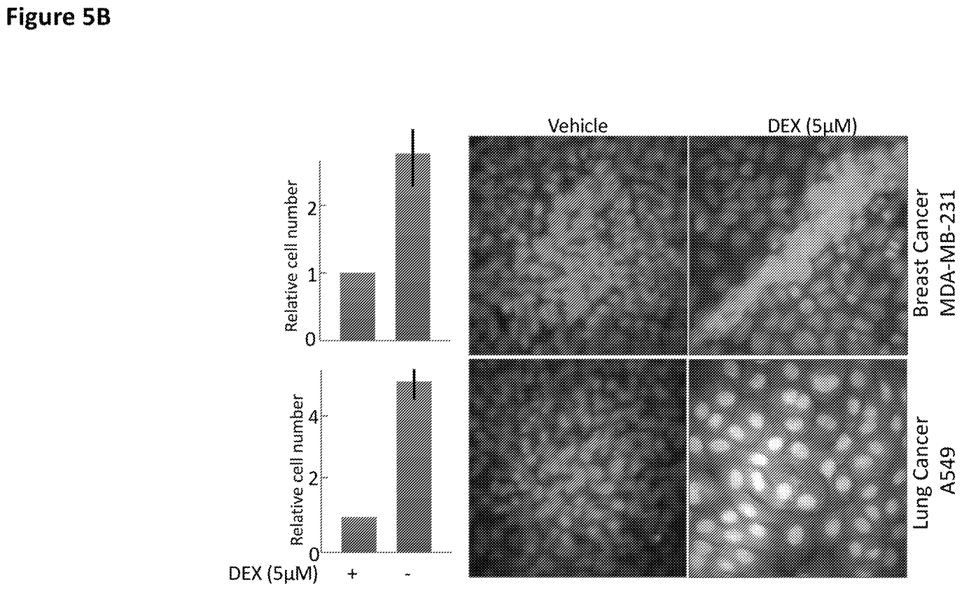

Reduction of final cell density and induction of vacuolation by HC and DEX were also observed in a murine lung cancer cell line TA2280 (FIG. 5A), a human lung cancer cell line A549, and a breast cancer cell line MDA-MB-231 (FIG. 5B). Despite that HC elicited variable efficiency among different cancer cell lines, these findings support that the contact inhibition restoration (CIR) activity of the two glucocorticoids is a general phenomenon and is not limited to a particular cell line or cancer type.

### Example 2

### Myc-Nick Promotes Contact Inhibition and Vacuolation

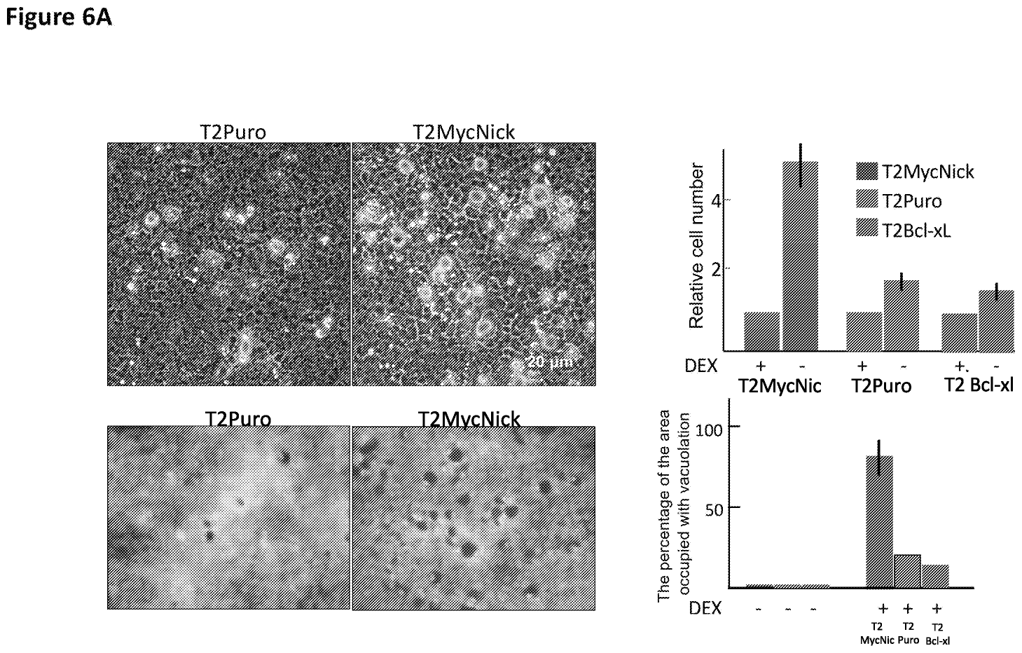

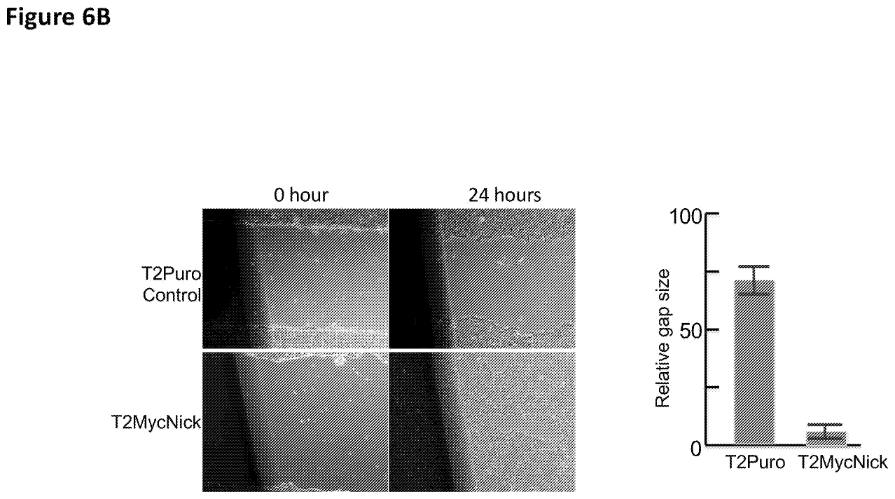

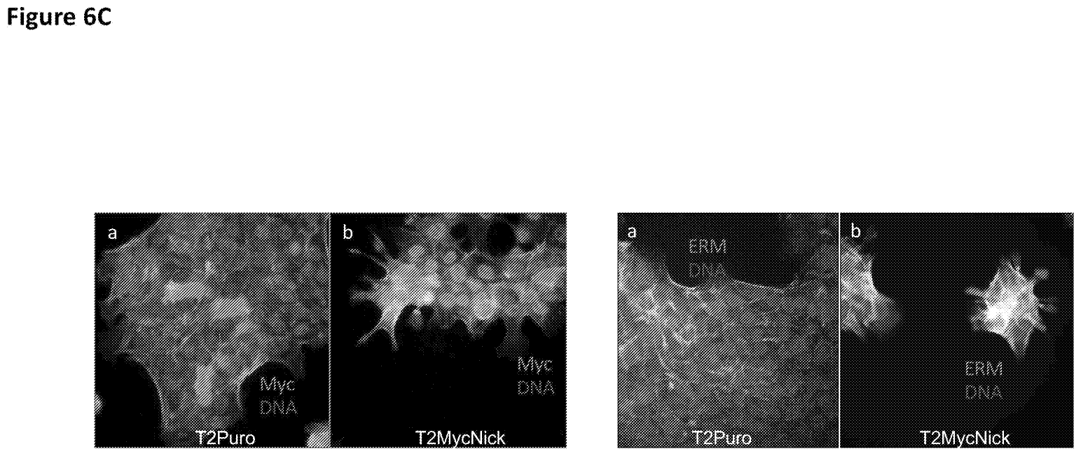

Ectopic expression of Myc-nick primed cells to the restoration of contact inhibition and induction of vacuolation by HC and DEX (FIG. 6A). T2Puro, a control cell line of T2MycNick cells that expresses a puromycin-selection marker gene, did not respond to HC and DEX. Likewise, T2Bc1-xL cells that express an antiapoptotic factor Bc1-xL in T2 cells did not confer sensitivity to HC and DEX. This contrast indicates that Myc-Nick but not Bc1-xL could prime cells to restore contact inhibition and induction of vacuolation in response to HC despite the observation that both Myc-nick and Bc1-xL could confer tumor cells with drug resistance. Consistent with a published study (Conacci-Sorrell et al., Cell 2010 Aug. 6; 142(3):480-93), ectopically expressed Myc-nick promoted cell migration in the wounding assay (FIG. 6B) and formation of filopodia (FIG. 6C), confirming that the expressed Myc-nick was functional. The HC-induced filopodia were enriched for Myc-nick and ERM proteins (FIG. 6C). These findings reveal a previously unknown activity for Myc-nick in collaborating with glucocorticoids in promoting both contact inhibition of proliferation (CIP) and gigantic vacuolation. These findings also show that Myc-nick can serve as a key predicative biomarker for the therapeutic efficacy of glucocorticoids against solid tumors.

### Example 3

### Hydrocortisone and Dexamethasone Elicit CIR Activity Through Their Canonical Target Glucocorticoid Receptor (GR)

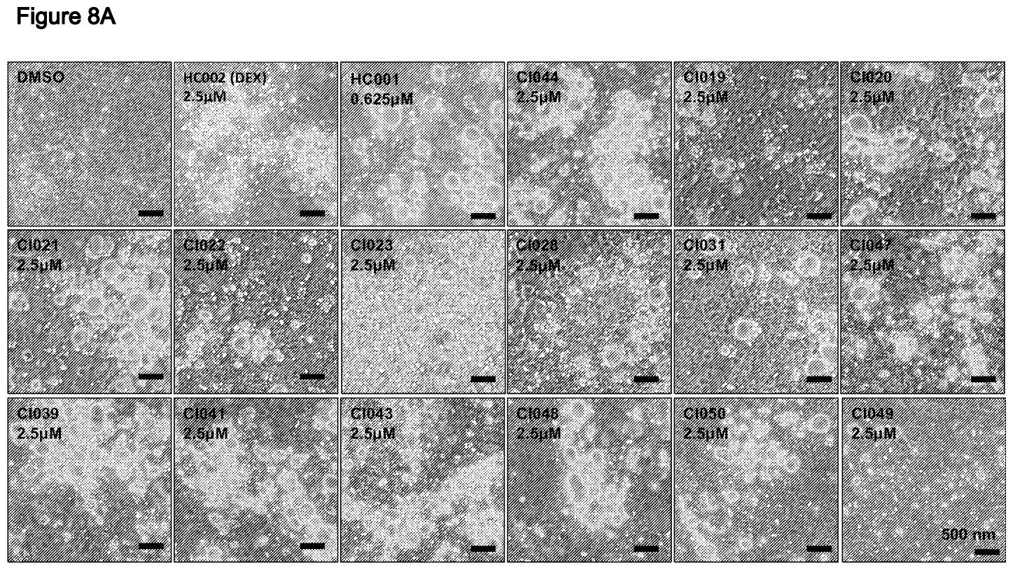

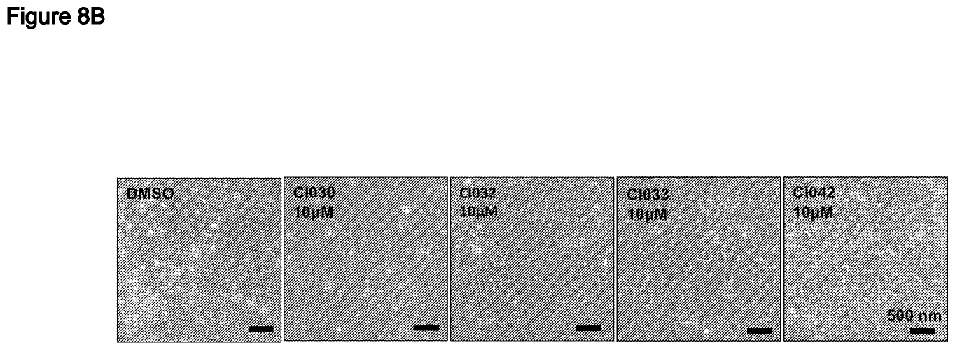

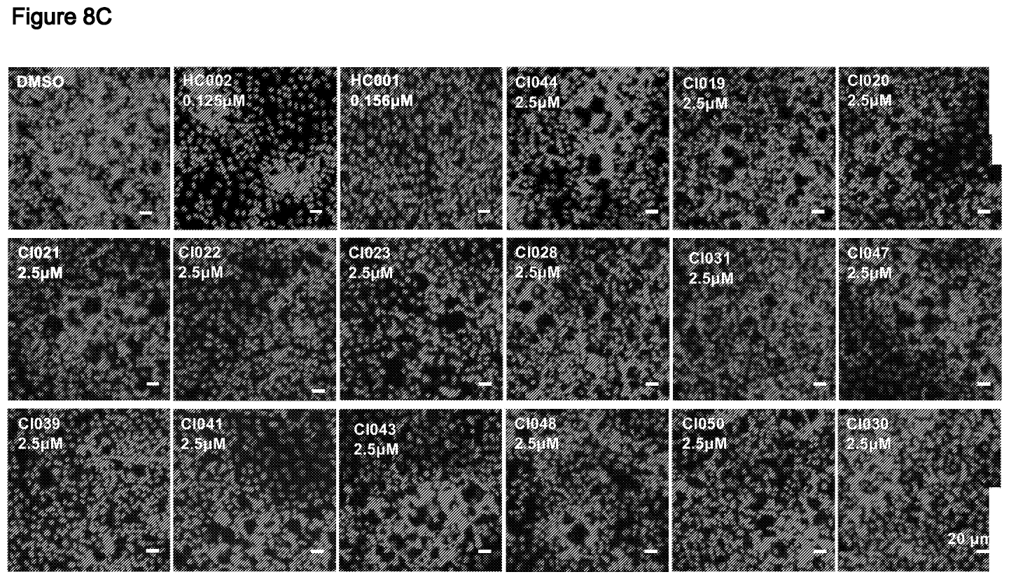

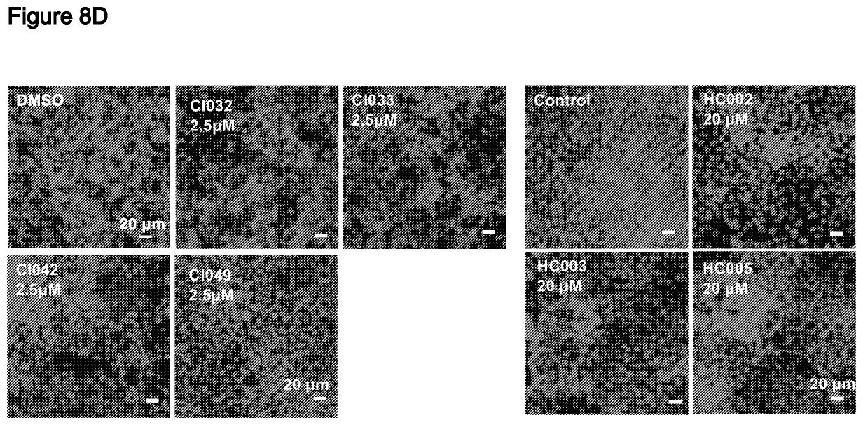

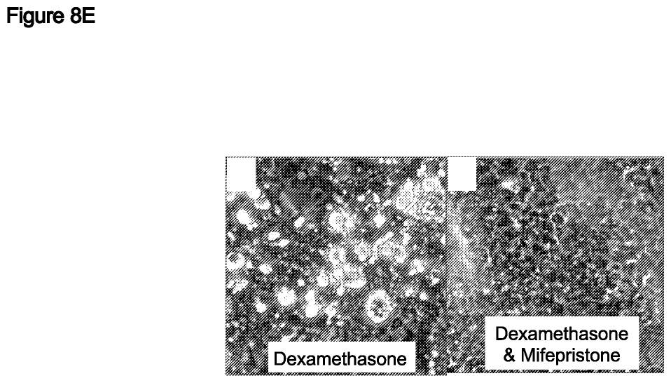

HC and DEX are known to act on the glucocorticoid receptor (GR) to elicit a variety of cellular activities. 23 additional glucocorticoid receptor agonists were tested in T2MycNick cells (FIG. 7) and the results were summarized in Table 1. Fifteen of the 23 glucocorticoids tested positive (FIGS. 8A-D). The remaining seven failed to elicit vacuolation even when used at a concentration up to 10 μM. Three out of the seven glucocorticoids reduced the cell density despite failing to elicit vacuolation (FIGS. 8A-D and Table 1). None of these compounds inhibited proliferation or induced cell death when MycNick cells were grown in sub-confluence. Three agonists of the progesterone receptor including ethynodiol diacetate, dienogest and ethisterone failed to elicit vacuolation or reduce cell density in T2MycNick cell line (Table 1 and FIG. 8). The CIR effect of HC was completely blocked by 5 μM of Mifepristone (RU486), an antagonist for both progesterone receptor (PR) and GR (FIG. 8E). Mifepristone, by itself, failed to elicit a CIR activity. Collectively, these data indicate that the CIR effect of HC and DEX was mediated by their well-characterized target GR.

TABLE 1

| | | Min. | Conc that | | | Min. | Conc that | | | Min. | Conc. that |
|---|---|---|---|---|---|---|---|---|---|---|---|
| | | effective | reduces > | | | Effective | reduces > | | | effective | reduces > |
| | | conc. that | 50% in | | | conc. that | 50% in | | | conc. that | 50% in |
| | | elicits | the cell | | | elicits | the cell | | | elicits | the cell |
| | | vacuolation | density | | | vacuolation | density | | | vacuolation | density |
| Number | Drugs | (μM) | (μM) | Number | Drugs | (μM) | (μM) | Number | Drugs | (μM) | (μM) |
| HC001 | Hydro- | 0.156 | 0.156 | CI019 | Beclo- | 0.625 | 2.5 | CI049 | Corto- | 5 | 10 |
| | cortisone | | | | methasone | | | | doxone | | |
| | | | | | dipropionate | | | | | | |
| HC002 | Dexa- | 0.125 | 0.125 | CI020 | Desonide | 0.625 | 2.5 | CI030 | Ethynodiol | >10 | 5 |
| | methasone | | | | | | | | diacetate | | |
| CI021 | Fluocinolone | 0.625 | 2.5 | CI028 | Cortico- | 0.625 | 2.5 | CI032 | Ethisterone | >10 | 10 |
| | Acetonide | | | | sterone | | | | | | |
| CI022 | Fluticasone | 0.625 | 2.5 | CI045 | Cortico- | 0.625 | 2.5 | CI033 | Dienogest | >10 | 10 |
| | propionate | | | | sterone | | | | | | |
| CI023 | Mometasone | 0.625 | 2.5 | CI047 | Triamcino- | 0.625 | 2.5 | CI042 | Prednisone | >10 | 2.5 |
| | Furoate | | | | lone | | | | | | |
| CI039 | Dexa- | 0.625 | 2.5 | CI048 | Fluocino- | 0.625 | 2.5 | HC004 | Hydro- | >10 | 10 |
| | methasone | | | | nide | | | | cortisone | | |
| | 21-phosphate | | | | | | | | 17- | | |
| | disodium salt | | | | | | | | Butyrate | | |
| CI041 | Triamcinolone | 0.625 | 2.5 | CI050 | Methyl- | 0.625 | 2.5 | HC003 | Hydro- | >10 | 2 |
| | acetonide | | | | prednisolone | | | | cortisone | | |
| | | | | | | | | | 21-acetate | | |
| CI043 | Fluoro- | 0.625 | 2.5 | CI031 | Medroxy- | 2.5 | 2.5 | HC005 | Dexa- | >10 | 10 |
| | metholone | | | | progesterone | | | | methasone | | |
| CI044 | Flumethasone | 0.625 | 2.5 | | acetate | | | | 21-acetate | | |

### Example 4

### Suppression of Tumorigenesis by CIR Compounds in 3-D Culture Assay and In Vivo Assay

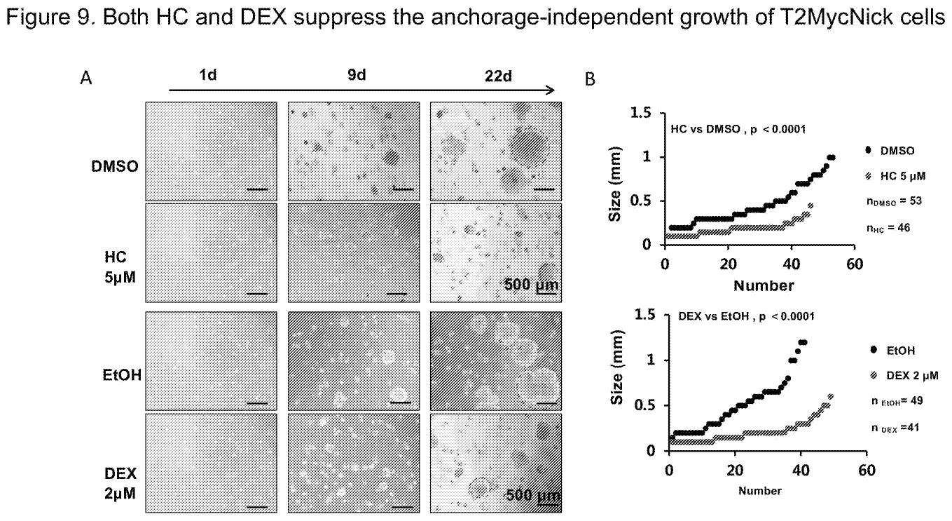

Growth of tumor cells in semi-solid soft agar is the gold standard for tumorigenesis in vitro and demands cells be able to divide in an anchorage-independent manner and override the contact inhibition of proliferation. The soft agar assay was performed as a secondary confirmatory assay for compounds identified for CIR. HC and DEX suppressed the anchorage-independent growth of T2MycNick cells (FIG. 9). It also demonstrated the therapeutic efficacy against tumorigenesis in xenograft assays (See below). Collectively, these findings validated the potential of the CIR platform in the discovery of oncology drugs that act by novel mechanisms.

### Example 5

### HC and DEX Trigger a Previously Undescribed Form of Cell Death Associated with Gigantic Vacuoles Only in Multilayer Zones

Glucocorticoids elicited the formation of vacuoles that emerged inside a cell, often expanded beyond its plasma membrane to contact neighboring cells, and peaked at a diameter of about 20 μm. Vacuoles identified in this study were much bigger than those reported in the literature and could reach a size larger than that of a cell. Induction of the vacuoles by HC and DEX occurred only when high-density cells were overgrowing. Induction of vacuolation did not occur in sub-confluent cells and occurred selectively in multilayer, not monolayer, zones of the cells treated with HC and DEX, indicating both cell-cell contact and cellular proliferation are utilized by glucocorticoids to elicit vacuolation.

Vacuolation has been known to be associated with several types of nonapoptotic cell death such as paraptosis, necrosis, lethal autophagy, and macropinocytosis (Yan et al., World Academy of Sciences Journal 2: 39-48, 2020). The vacuoles triggered by HC and DEX were not traditional autophagic vesicles because they were negative for ATG8/LC3, a resident protein of autophagosomes and autolysosomes (Xie Z. and, Klionsky D J, Nat Cell Biol 9:1102-1109, 2007). Some chemicals have been reported to trigger nonapoptotic cell death associated with accumulation of vacuoles due to enlargement of endoplasmic reticulum (Sharma et al., Scientific Reports vol. 11, Article number: 30899 2021); (Lee W. et al., Scientific Reports, 27 May 2015, 5:10420). The peripheral membrane of HC-induced vacuoles tested negative for an ER marker Calnexin or a Golgi marker Golgin 97, indicating the origin of the vacuoles were not likely due to fragmentation and enlargement of either ER or Golgi. Macropinocytosis promoted by an active RAS in a glioblastoma cell line is a mechanism by which cells ingest extracellular fluid and its contents through the formation of invaginations by the cell membrane, which close and break off to form fluid-filled vacuoles in the cytoplasm (Ramirez, C. et al., Nature 2019 December; 576 (7787):477-481). Vacuoles associated with macropinocytosis do not sequester organelles or cytoplasm and are not acidic. However, HC-induced vacuoles did sequester cytoplasmic components and nuclei. Furthermore, paraptosis inhibitors such as DPP4 inhibitors—linagliptin and alogliptin failed to inhibit HC-triggered vacuolation.

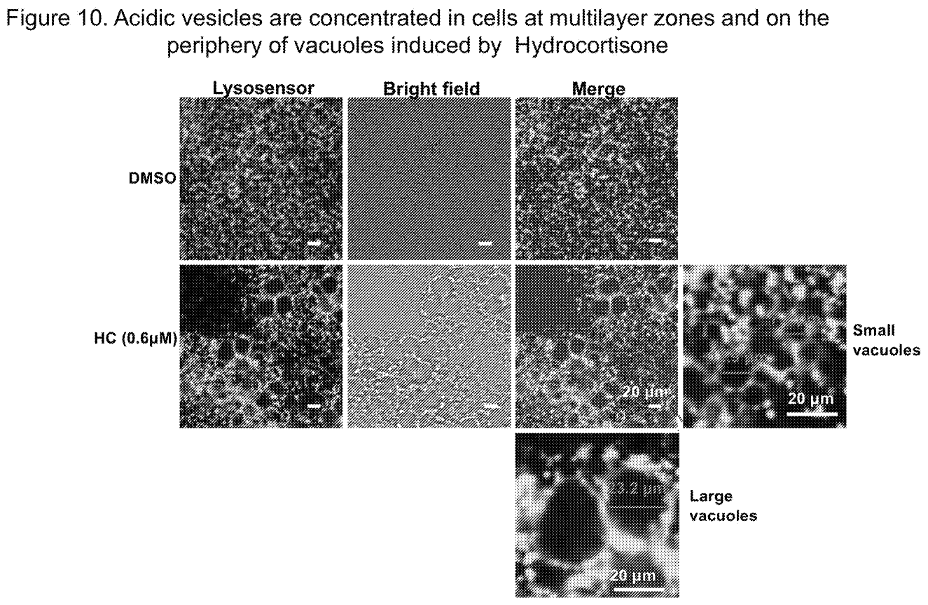

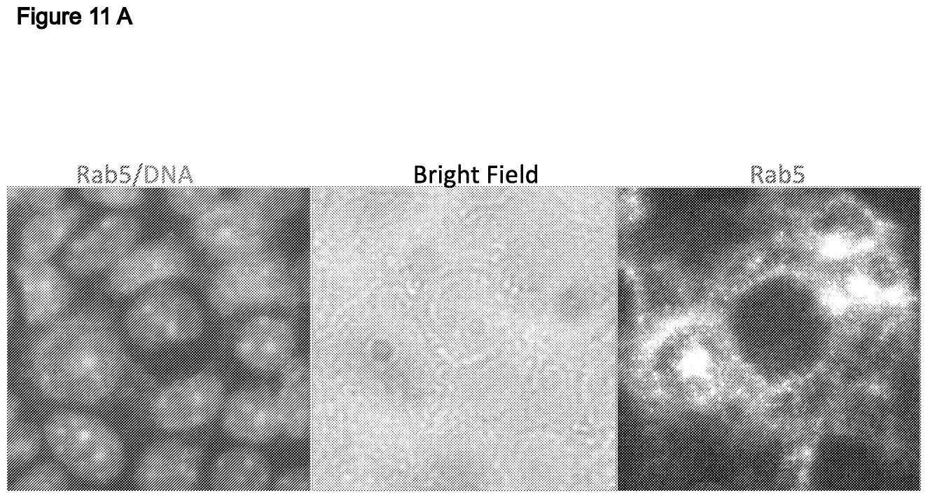

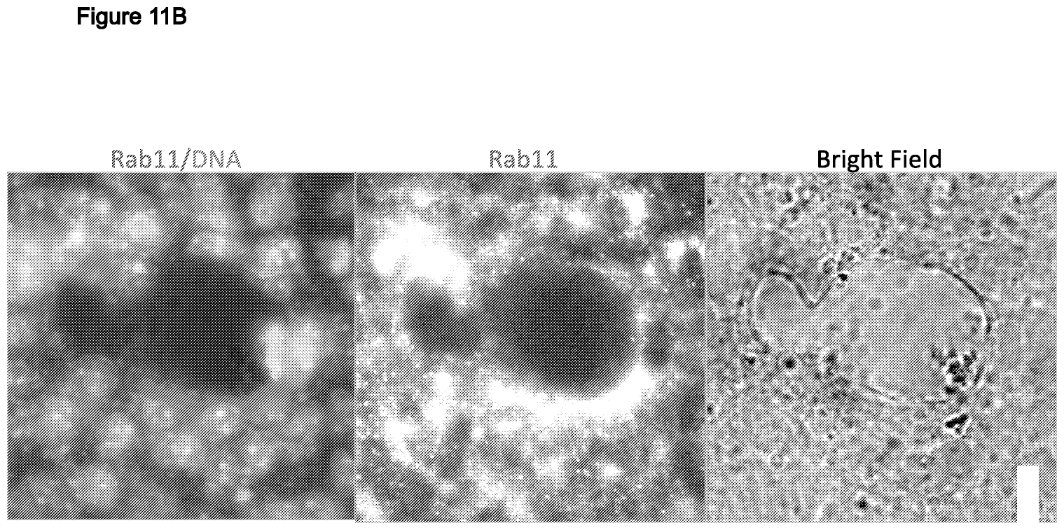

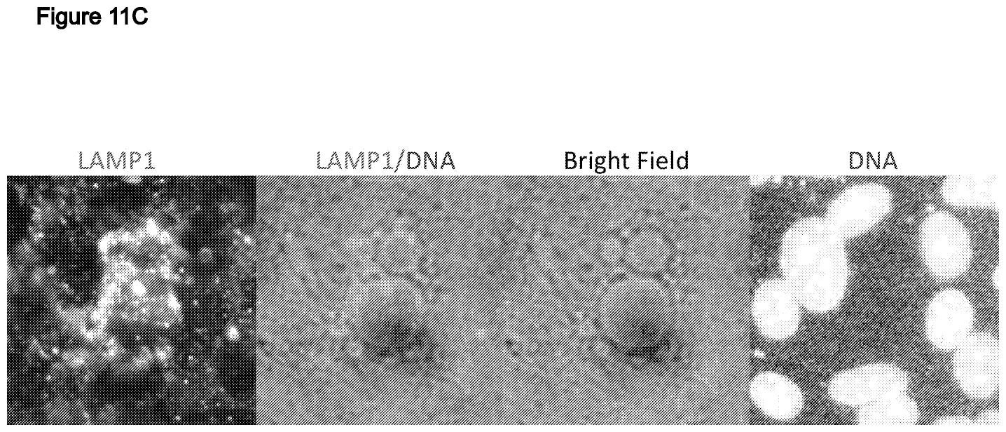

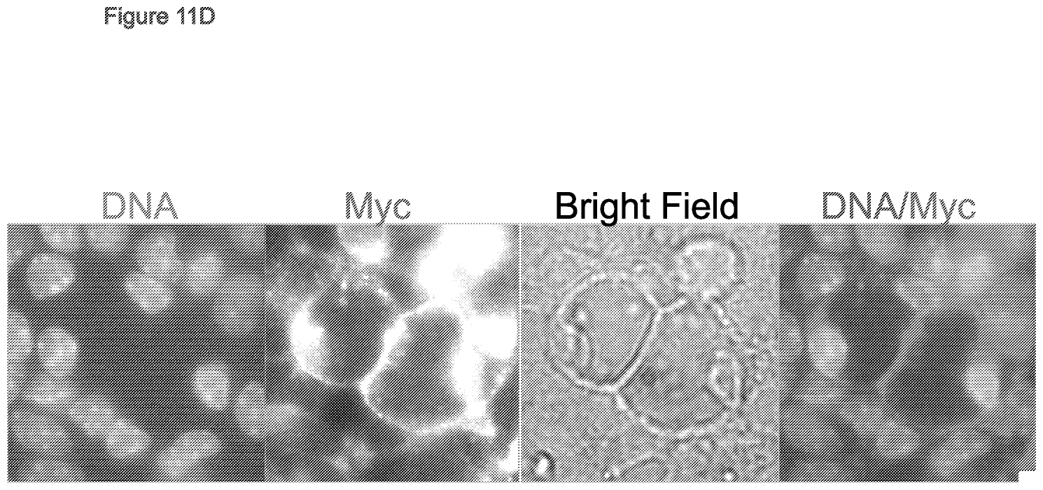

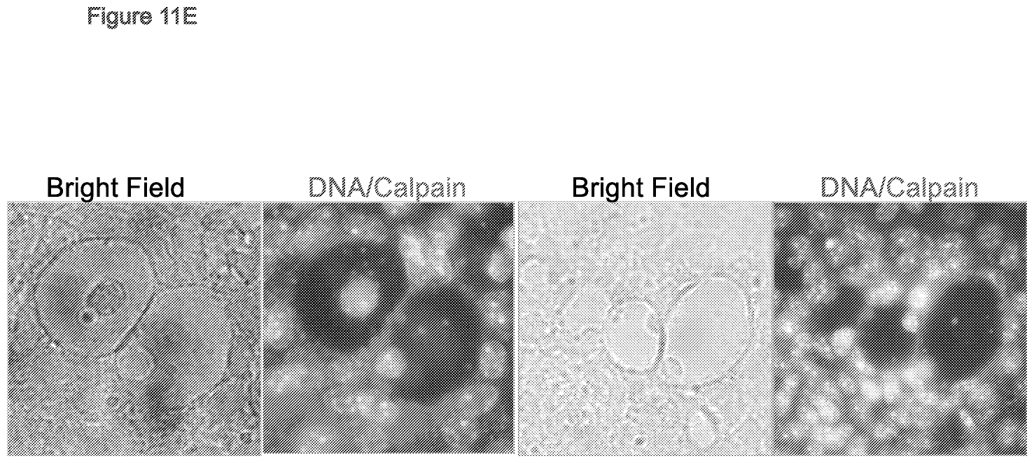

Small acidic vehicles labelled with acridine orange and lysosensor green DND-189 highly accumulated on the HC-induced vacuolar membrane and in the cells near a vacuole (FIG. 10). The peripheral membrane of each vacuole was unevenly loaded with components of endosomes and lysosomes, including an early endosome marker, Rab5, a recycling endosome marker, Rab11, a lysosome marker, LAMP1, clathrin, and the master regulator of lysosome biogenesis, TFEB (FIGS. 11A-C and data not shown). Thus, endosomes, lysosomes and the intracellular membrane trafficking system were indicated to be involved in vacuolation elicited by glucocorticoids.

A calcium-activated cytoplasmic protease, calpain, and Myc-nick were also highly enriched on the vacuolar membrane and in cells surrounding vacuoles (FIGS. 11D-E). Combined with the report that Calpain is known to cleave the full-length Myc into Myc-nick (Conacci-Sorrell et al., Cell 2010 Aug. 6; 142(3):480-93), these findings implicate the Calpain-Myc-nick axis in the responses to glucocorticoids. Therefore, both abundant Myc-nick and high calpain activity might be useful biomarkers that predict a favorable CIR response to glucocorticoids.

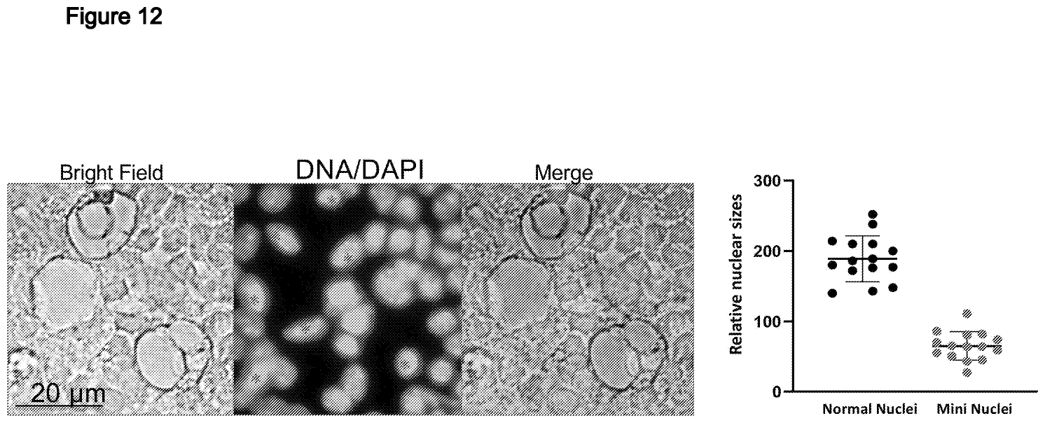

The content inside of vacuoles: The inside of the vacuoles was largely electron-translucent and filled with fluid and 3-5 small aggregates (FIGS. 11 and 12). Occasionally, electron-dense solid debris, small vacuoles, and condensed nuclei were seen within a vacuole. Small acidic vehicles labelled with acridine orange and lysosensor green DND-189 highly accumulated on these solid objects.

Nuclear condensation: HC elicited nuclear condensation in cells located in multilayer but not monolayer zones. The sizes of condensed nuclei ranged from one half to less than one tenth of normal nuclear sizes (FIG. 12). Cells with a highly condensed and miniature nucleus were presumably dead even though they were negative for active caspases and were not permeable to Trypan blue or PI. These mini nuclei appeared coincidentally with the emergence of glucocorticoid-induced vacuoles both spatially and temporarily. Multiple mini nuclei were typically found at the inner or outer periphery of each vacuole (FIG. 12). Occasionally, a compressed nucleus was found at the boundary of two adjoining vacuoles. In each case, vacuole-associated nuclei were highly condensed, in particular at the nuclear edge that was in contact with the vacuolar membrane. The merging area and mini nuclei were frequently enriched for lysosomes, calpain and other the markers mentioned above, to the point of being at their highest activity in these areas of drug-treated cells. These results indicate that condensed, mini nuclei likely resulted from the action of lysosomal and cytoplasmic digestion enzymes.

Glucocorticoid-induced vacuolation as a new type of nonautonomous cell death: These results also suggest that glucocorticoid-induced vacuoles actively degrade cytoplasmic components and nuclei by recruiting both lysosomal enzymes and the cytoplasmic protease such as calpain. Apparently, this type of cell death is not self-constrained within a cell and instead often extends to neighbors because one vacuole is typically associated with multiple condensed mini nuclei. This nonautonomous cell death occurred only in multilayer zones but not in monolayer zones. Neither HC nor DEX elicited such changes when the same cells were cultured in sub-confluence. This form of nonapoptotic cell death and gigantic vacuoles have not bene previously described in the literature.

Preferential labeling of the vacuolar membrane and condensed mini nuclei by CellTracker Green CMFDA (5-chloromethylfluorescein diacetate). CMFDA is an uncharged, non-fluorescent, lipid-soluble and reactive dye that is hydrolyzed to fluorescein by nonspecific intracellular esterases after uptake. Free fluorescein is polar and retained by intact cells to levels of measurable fluorescence. CMFA also contains a chloromethyl group that reacts with thiol groups, which can be catalyzed by a Glutathione-S-transferase. In most cells, glutathione levels are high (up to 10 mM) and glutathione transferase is ubiquitous.

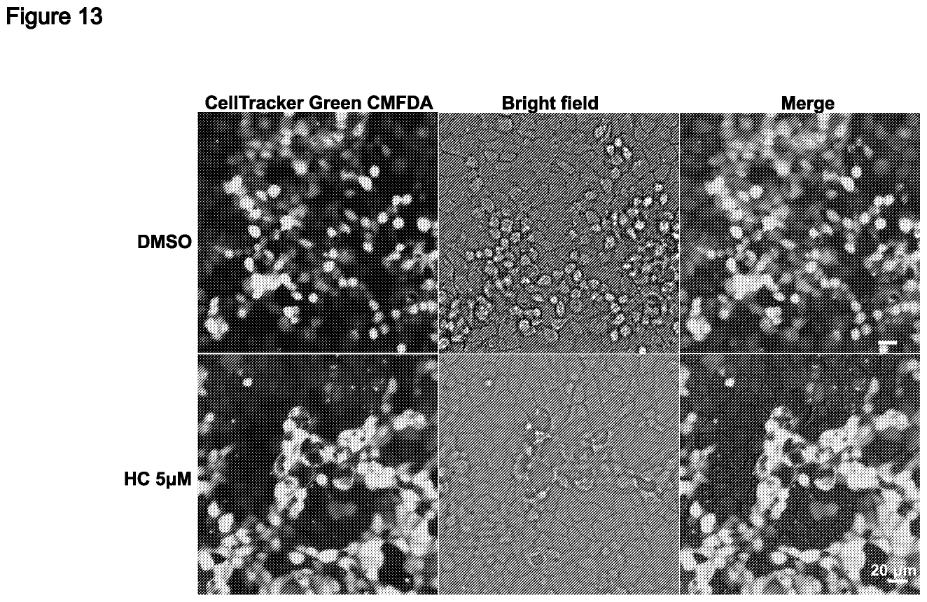

As expected, the CMFA dye strongly labelled cytoplasm in T2MycNick cells treated with DMSO (FIG. 13). In T2MycNick cells treated with HC, CMFA preferentially accumulated in multilayer zones as opposed to monolayer zones (FIG. 13). In multilayer zones, vacuolar membrane and condensed mini nuclei associated with vacuoles were preferentially labelled by CMFDA. Except for condensed mini nuclei, the other inside content of vacuoles was largely not labelled by the dye.

CMFA selectively labelled HC-induced vacuolar membrane as opposed to single membrane of subcellular organelles and double membranes of cytoplasm. This selectivity implies that HC-induced vacuolar membrane harbors a unique component that can be conjugated with CMFA or catalyze such a bioconjugation reaction. The selectivity also implies that HC-induced vacuoles may not originate from existing membranes in cells. Instead, it is likely synthesized de novo, with a unique component that is absent in any of the other membranes.

CFMA preferentially labelled the condensed mini nuclei associated with vacuoles but not normal nuclei in either multilayer or monolayer zones. The hypothesized CFMA-conjugation target or enzyme could also be acquired by nuclei undergoing the HC-induced nonapoptotic cell death. A condensed nucleus might acquire this feature through its contact with the vacuolar membrane.

These results indicate that CFMA can be a useful probe to detect vacuoles and condensed mini nuclei during the lysosome-associated nonapoptotic cell death elicited by glucocorticoids.

### Example 6

### HC and DEX Restore Cell-Cell Junctions Only in Monolayer Zones

HC and DEX not only restored contact inhibition but also cell-cell junctions in monolayer zones. Cell-cell junctions in multilayer zones were not detected. A similar relocation in response to glucocorticoids in T2MycNick cells was observed.

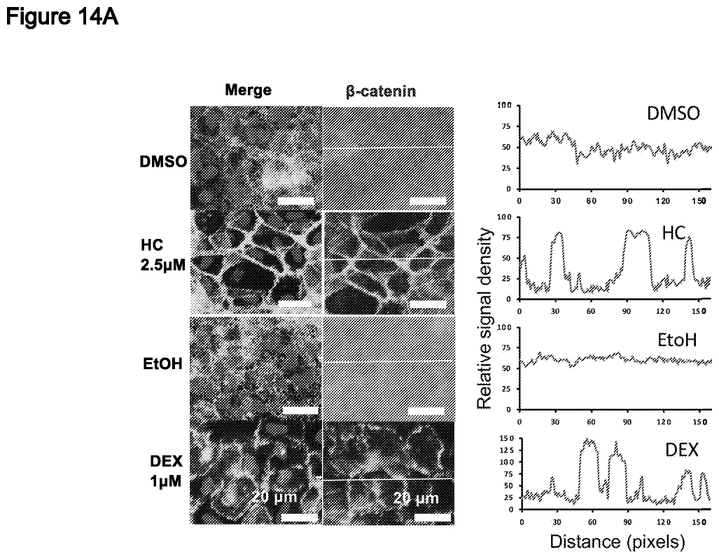

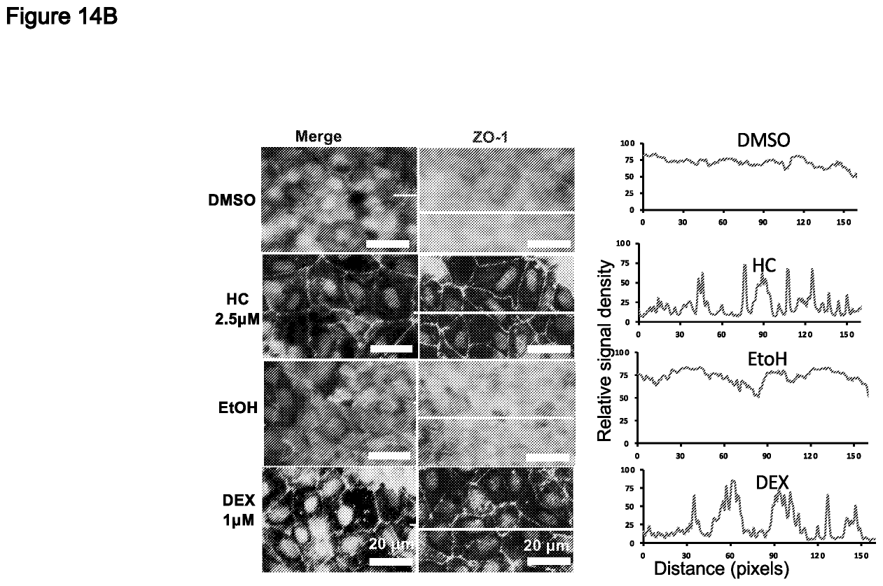

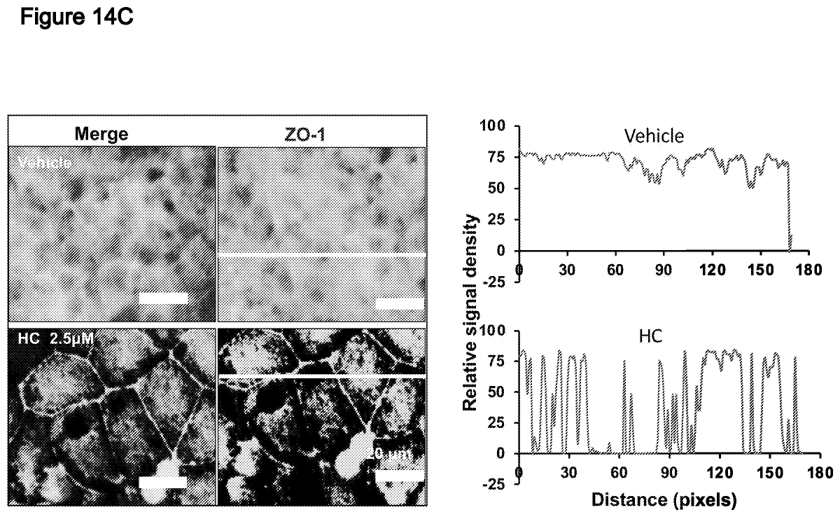

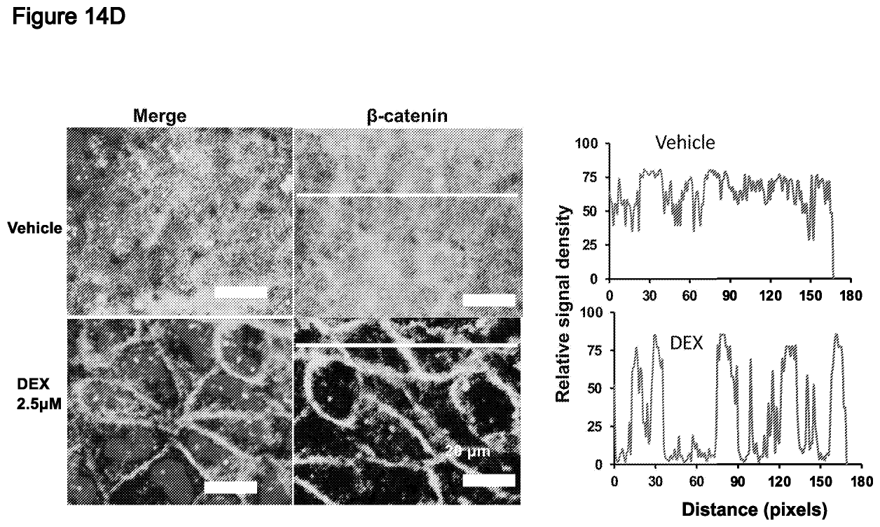

T2MycNick cells in monolayer zones displayed ZO1 and β-catenin predominantly localized to the lateral cytoplasmic membrane (FIGS. 14A-B). In contrast, both proteins largely occupied the cytoplasm and nuclei in cells located in multilayer zones or cultured under sub-confluence. Different from the findings in T2MycNick cells, treatment of T2Puro cells failed to induce such relocation. These findings show that MycNick was used by HC to establish cell-cell junctions.

Similar to the findings in liver cancer cell line T2MycNick, HC and DEX could model overgrown lung cancer cell line TA2280 in multilayers into localized monolayers of confluent cells. Although Myc-nick was not ectopically expressed in TA2280, which has abundant expression of Myc, the relocation of ZO1 and β-catenin to the lateral membrane occurred in the monolayer zones (FIGS. 14C-E). Endogenous Myc-nick in confluent cells might collaborate with HC to relocate ZO1 and β-catenin to the lateral cytoplasmic membrane. Thus, the role of glucocorticoids in promoting cell-cell junction localization of these two proteins is likely a general phenomenon and is associated with the formation of monolayer cells of confluence.

Cell-cell contact in multilayer zones was not sufficient to allow HC and DEX to induce localization of ZO-1 and β-catenin to the plasma membrane. In contrast, lateral membrane localization of these two proteins was uniformly observed in monolayer zones, which were proliferatively arrested. This contrast indicates that proliferative arrest might be a predeterminant used to establish cell-cell junctions. Inhibition of proliferation, however, was not sufficient to induce the formation of cell-cell junctions since an arrest of proliferation in a high-density cell population by deprivation of serum did not lead to the re-localization of either ZO-1 or β-catenin to the lateral cell membrane. These results show that glucocorticoids directly regulate both CIP and cell-cell adhesion.

### Example 7

### Suppression of Tumorigenesis by HC and DEX

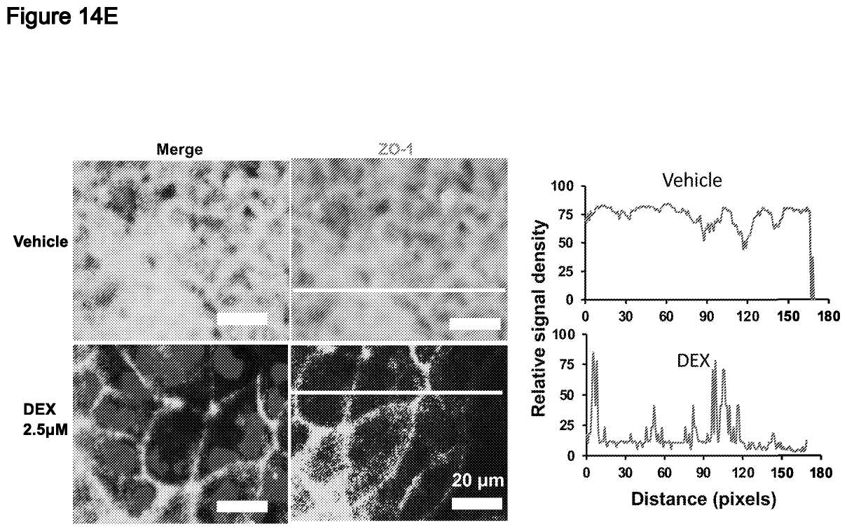

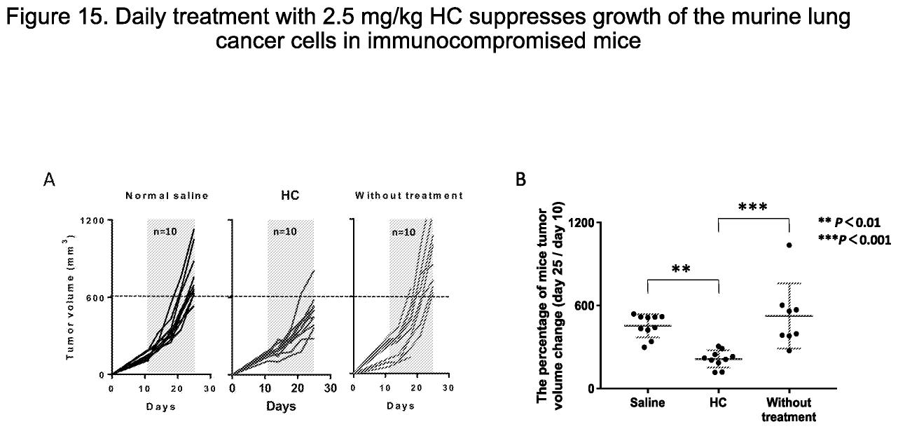

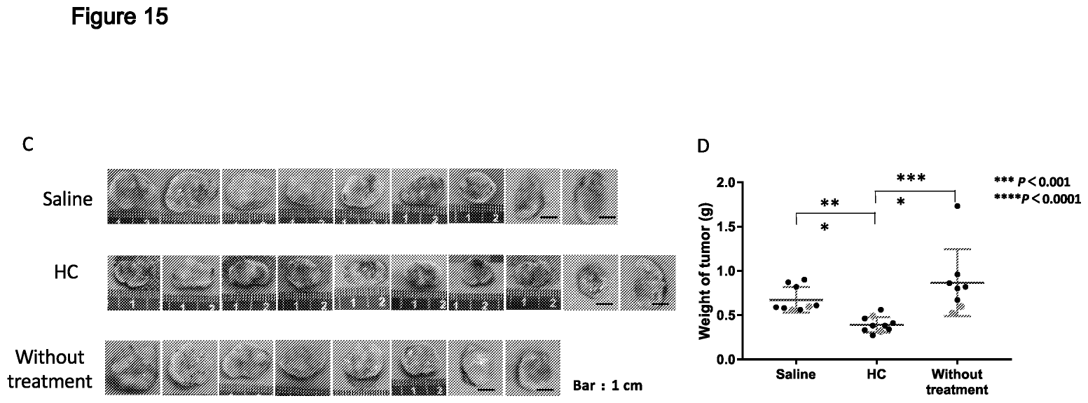

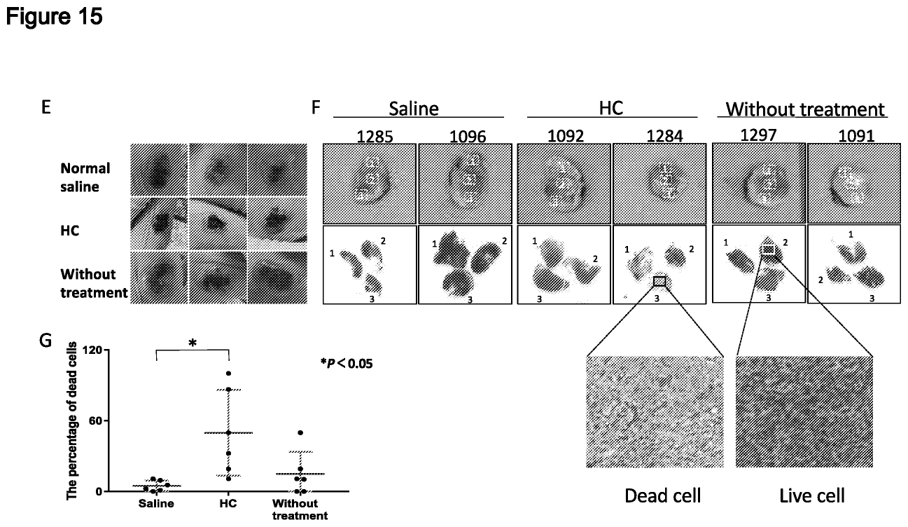

Consistent with its effect on cells growing in 2D culture, HC and DEX suppressed the 3D growth of the MycNick cancer cells in soft agar (FIG. 9). The therapeutic efficacy of HC was examined in a randomized, double-blinded, and placebo-controlled clinical trial in mice. HC administered at a daily dose of 2.5 mg/kg markedly suppressed the growth of mouse lung cancer cells driven by BRAF^{V600E} and liver cancer cells driven by MYC (FIGS. 15A-D). In size-matched tumors between control group and treatment group, histological analysis of tumor tissues revealed that HC elicited massive killing of tumor cells, indicating that HC suppressed tumor growth primarily by induction of cell death in vivo (FIGS. 15F-G). This treatment was well tolerated by mice and had no obvious effect on general health. These findings indicate that drugs that do not have apparent cytotoxicity against sub-confluent cells in vitro could still elicit massive killing of tumor cells in vivo. This is also the first example that contact inhibition of cancer cells could be restored by chemicals in vitro and that a CIR compound could exert therapeutic efficacy in vivo.

### Example 8

### Calcineurin Inhibitors Enhance the Effect of HC in Restoration of Contact Inhibition and Suppression of Tumorigenesis

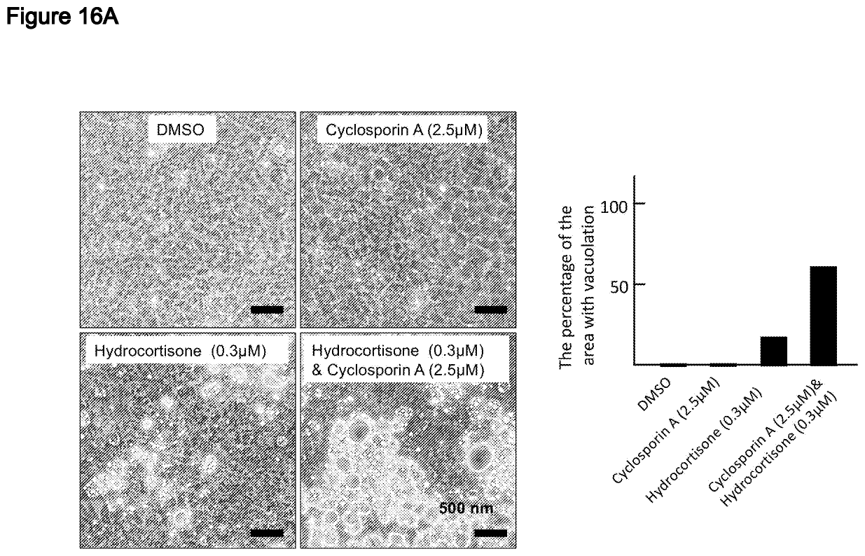

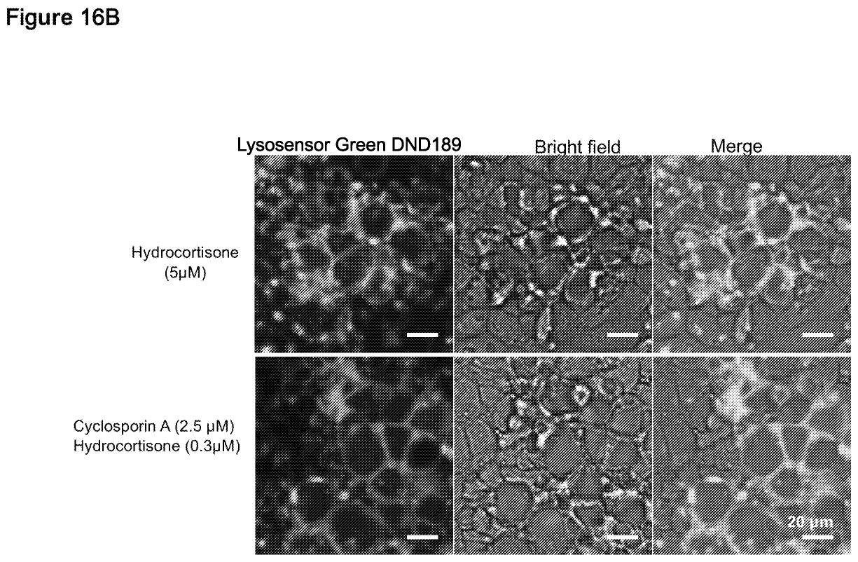

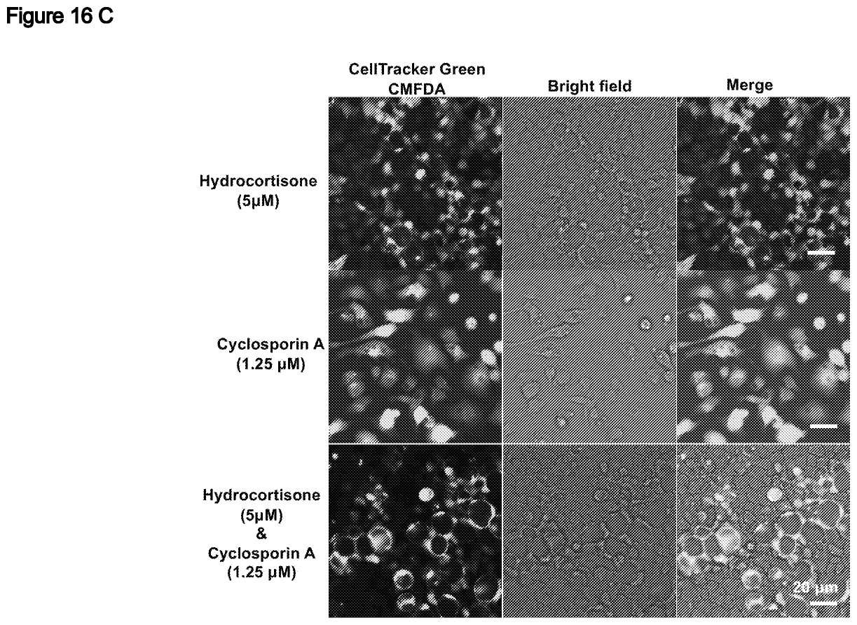

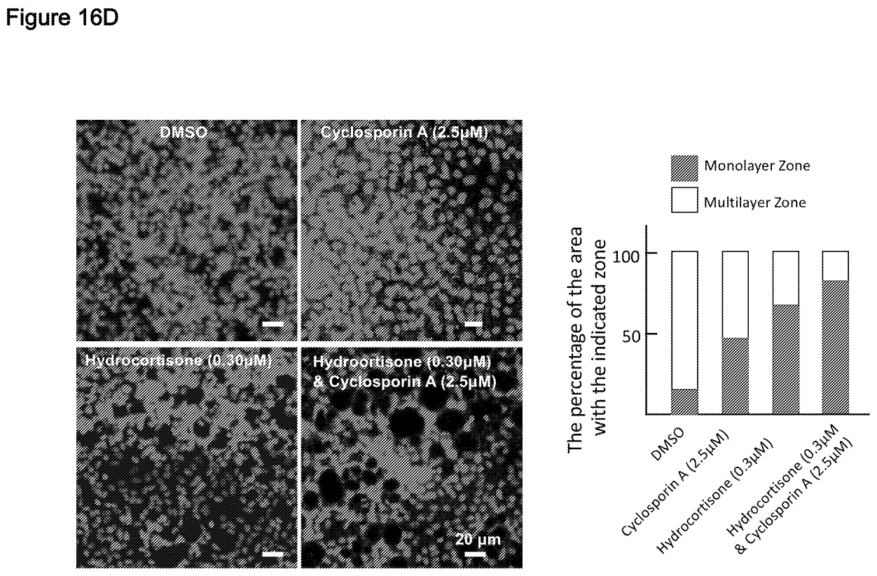

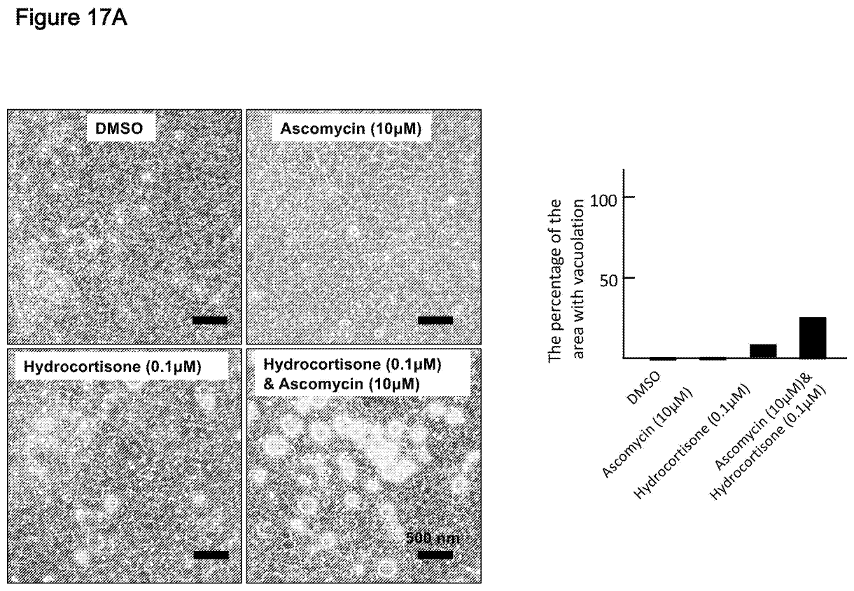

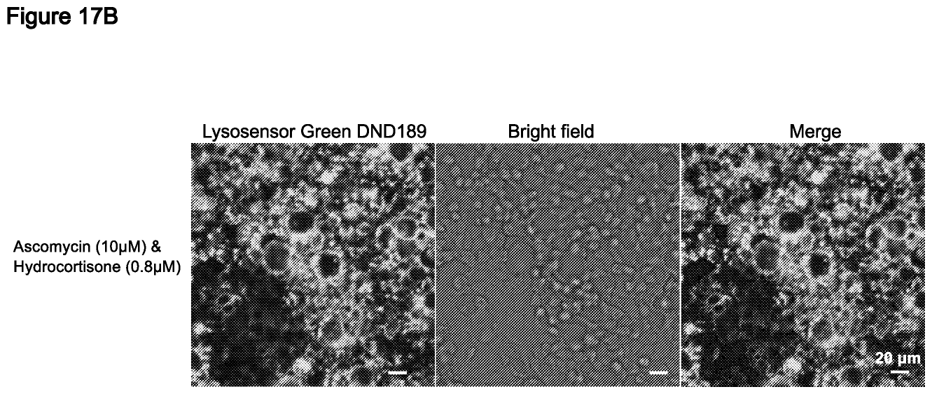

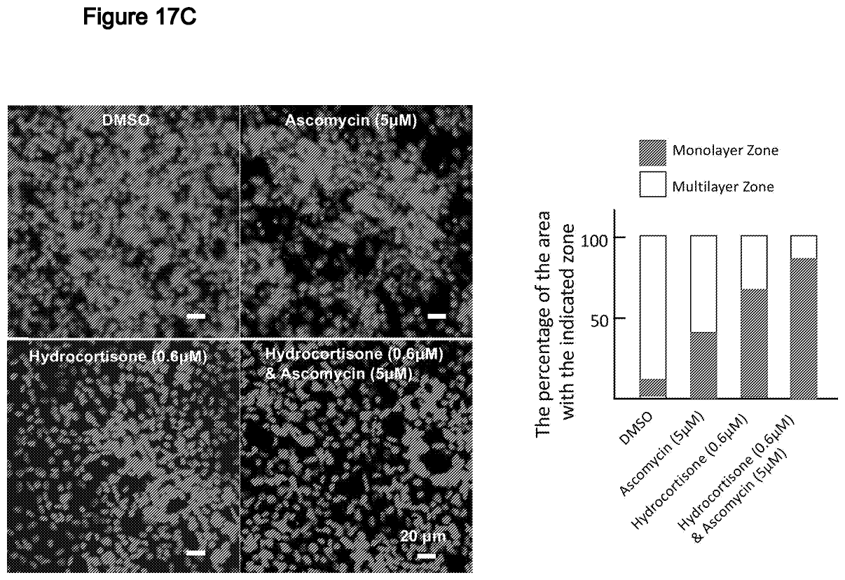

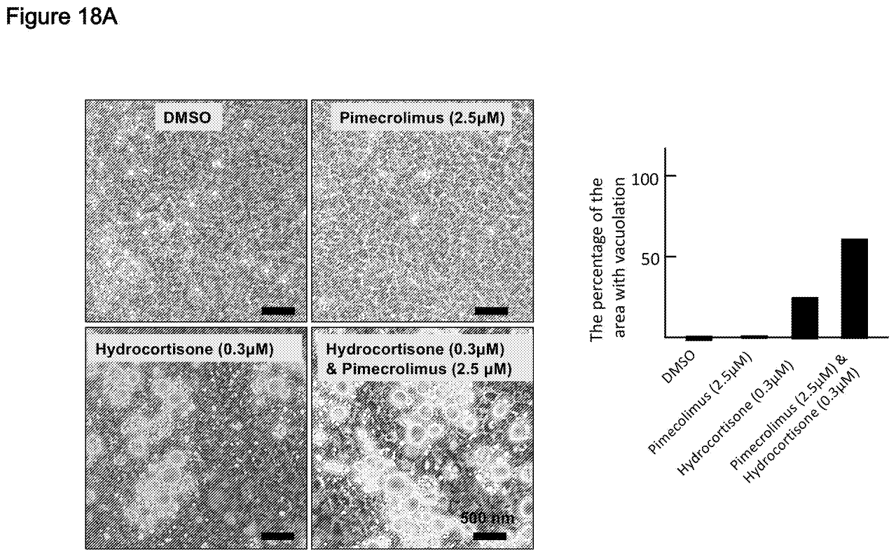

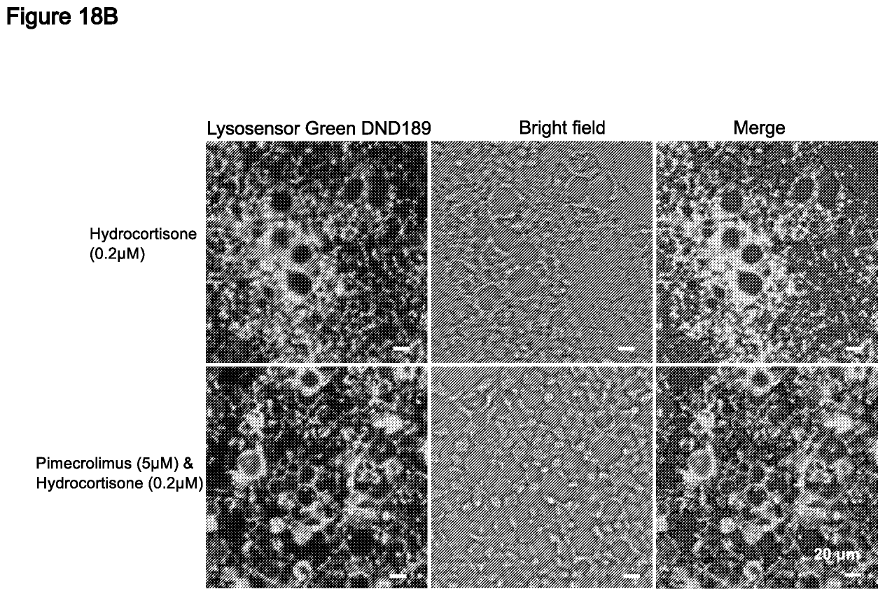

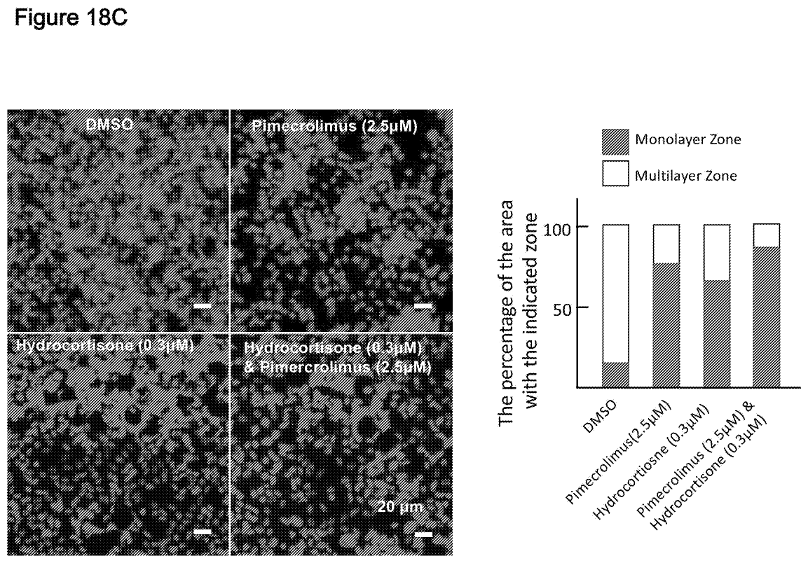

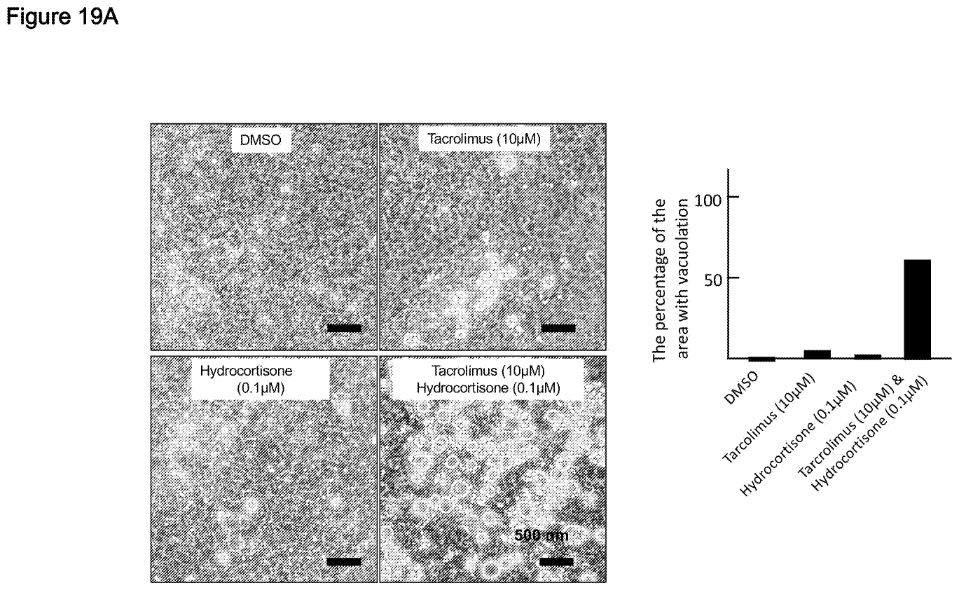

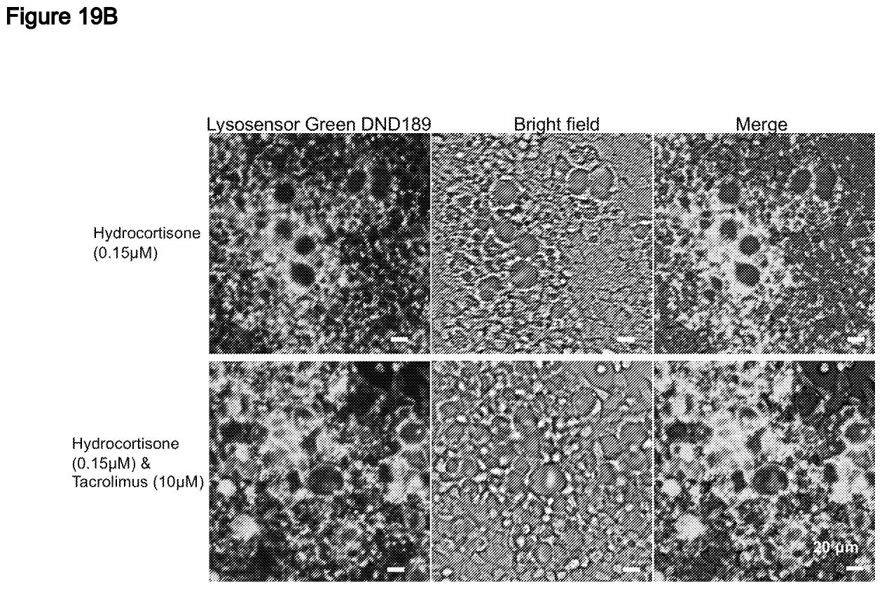

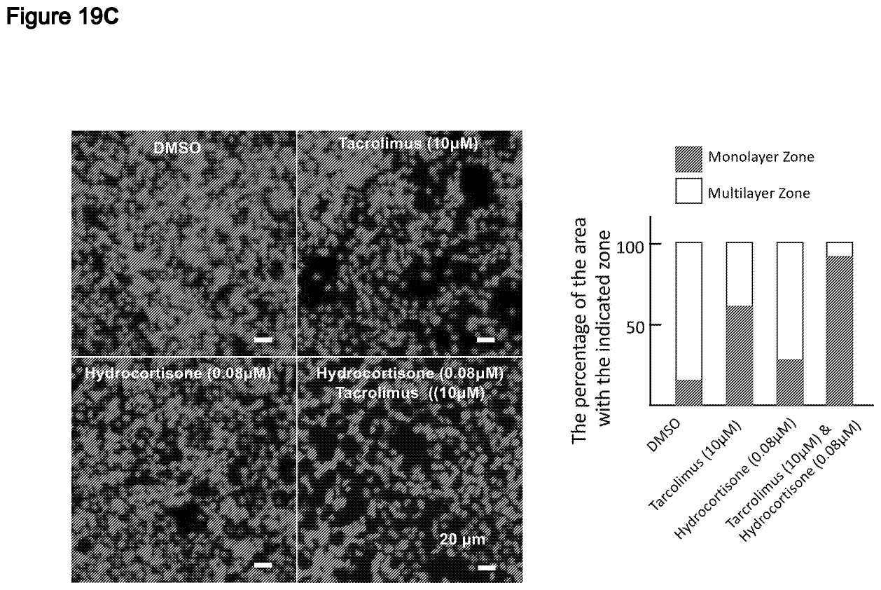

A chemical library for enhancers of the CIR effect of HC in MycNick cells was screened. Four calcineurin inhibitors were identified including Cyclosporin A (CsA), Ascomycin, Pimecrolimus and Tacrolimus (Table 2). Although these calcineurin inhibitors did not elicit vacuolation by themselves at the concentration tested in this study, each of them primed cells to develop extensive vacuolation in response to otherwise ineffective, low concentrations of HC (FIGS. 16A, 17A, 18A, and 19A).

The enhanced vacuoles were similar to those elicited by high concentration of HC alone with regard to sizes, morphology and association with acidic vesicles (FIGS. 16A-B, 17A-B, 18A-B and 19A-B). Like vacuoles elicited by HC, vacuoles enhanced by CsA were also preferentially labelled by CellTracker Green CMFDA dye (FIG. 16C).

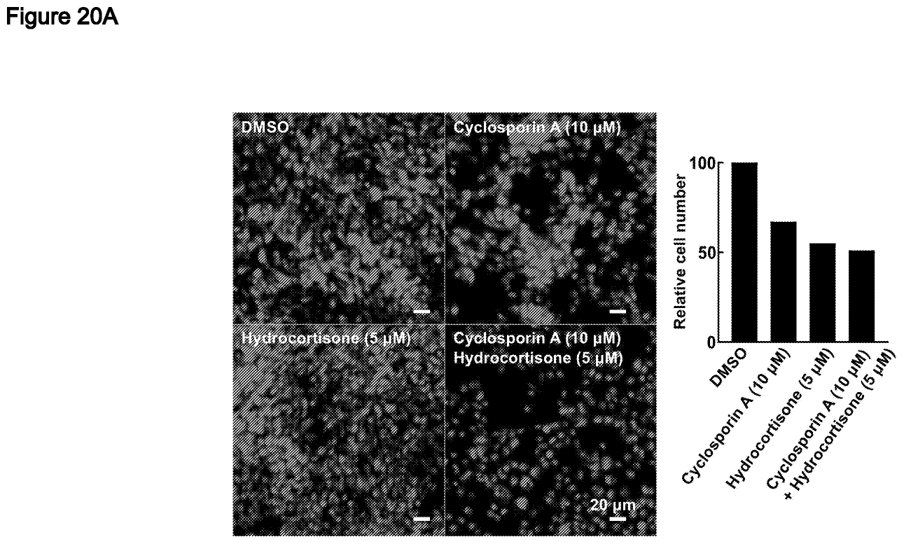

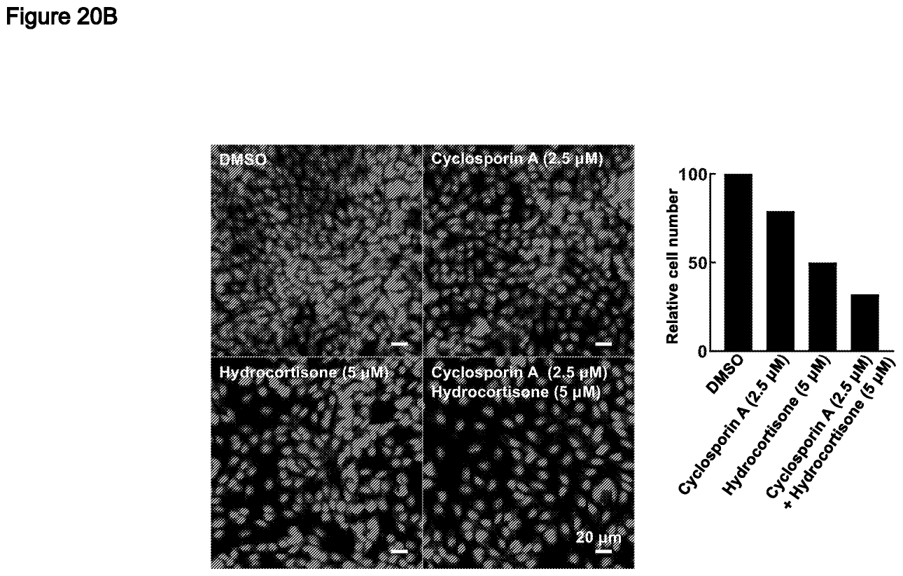

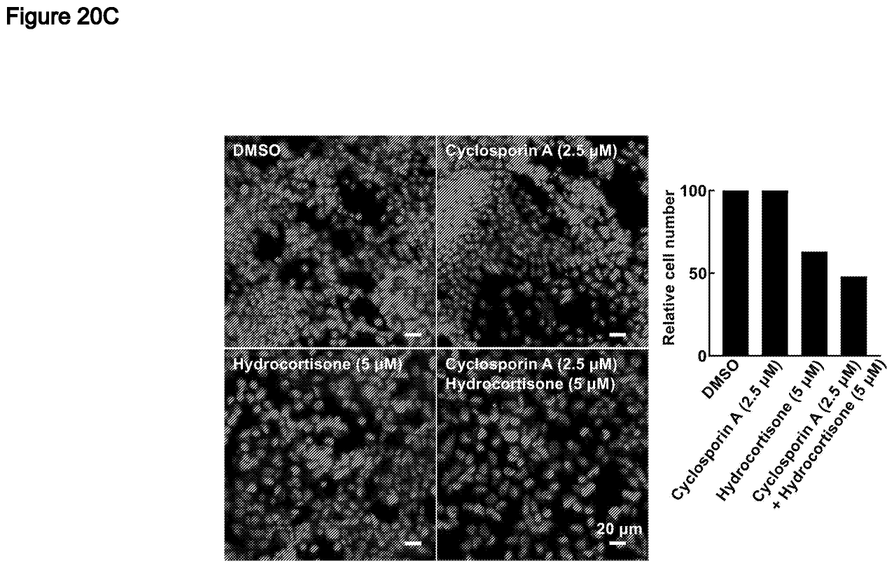

Combined treatments were also more potent than mono-treatments in reducing the cell density when T2MycNick cells were allowed to grow beyond confluence (FIGS. 16D, 17C, 18C and 19C). Similar results were observed with a BRAF^{V600E}-driven mouse lung cancer cell line MM (FIG. 20A), a human lung cancer cell line A549 (FIG. 20B), and a MYC-driven mouse liver cancer cell line (FIG. 20C). Combined treatments reduced the cell density to a greater extent than either single treatment in each of the above cell lines.

Rapamycin (Rap), FK506 and FK520 are structurally related compounds that have a common structural motif, which is responsible for their specific binding to FK506-binding proteins (FKBPs) such as FKBP12. Their distinct biological effects arise from their subsequent binding to different downstream protein targets. The RAP-FKBP12 complex inhibits the mammalian target of rapamycin (mTOR), but does not inhibit calcineurin. Both FK506-FKBP12 complex and FK520-FKBP12 complex inhibit calcineurin but not mTOR. In contrast to the calcineurin inhibitors, Rap failed to enhance the effect of HC in eliciting vacuolation. Conversely, it blocked the ability of HC in eliciting vacuolation. This contrast provided further evidence that inhibition of calcineurin sensitized cells to induction of vacuolation by HC. mTOR activity was involved in the formation of vacuoles.

TABLE 2

| | | Combination treatment with | |
|---|---|---|---|
| | | Hydrocortisone for 7 days | |
| | | Vacuolation by | Cell density reduction |
| Number | Drugs | Hydrocortisone | by Hydrocortisone |
| CI055 | Cyclosporin | Enhanced | Enhanced |
| | A | | |
| CI034 | Ascomycin | Enhanced | Enhanced |
| CI040 | Pimecrolimus | Enhanced | Enhanced |
| CI046 | Tacrolimus | Enhanced | Enhanced |

### Example 9

### CsA and HC Act Synergistically in Suppressing Tumorigenesis Driven by BRAF^{V600E}

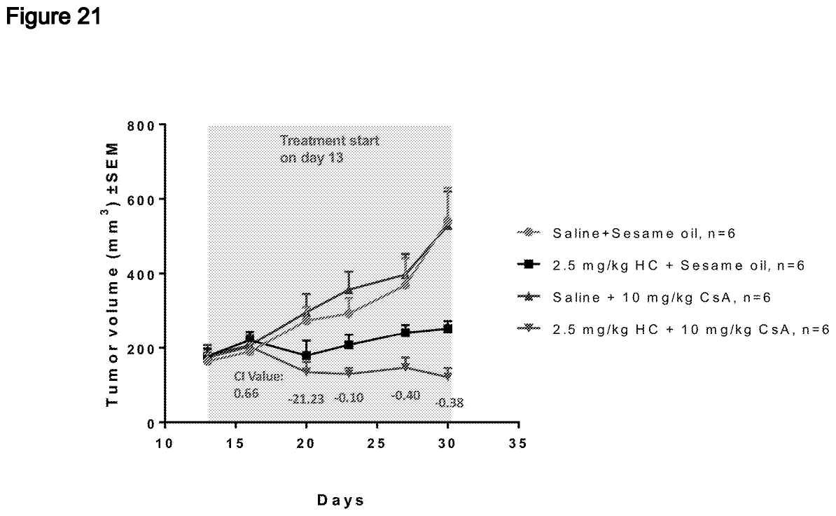

Nu/Nu mice were implanted with the murine lung cancer cell line harboring the BRAF^{V600E} mutation. When the average tumor size reached 200 mm³, tumor-bearing mice were randomized to receive daily treatment with Hydrocortisone and Cyclosporin A either individually or in combination. Tumor sizes were measured with a caliper once every three days (FIG. 21). These results show that Hydrocortisone and Cyclosporin A synergistically suppress tumor growth.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of treating cancer in a subject, comprising administration to the subject of (1) one or more glucocorticoid receptor agonists and (2) one or more inhibitors of calcineurin.

2. A method of reducing cancer cell proliferation, comprising administration to a subject (1) one or more glucocorticoid receptor agonists and (2) one or more inhibitors of calcineurin; wherein the cell proliferation is reduced compared to cancer cells that have not been contacted with the one or more glucocorticoid receptor agonists and the one or more inhibitors of calcineurin.

3. A method of reducing cancer cell proliferation in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin.

4. A method of inducing vacuolization of cancer cells, comprising administration to a subject (1) one or more glucocorticoid receptor agonists and (2) one or more inhibitors of calcineurin; wherein the vacuolization is characterized by an increase in the presence of vacuoles compared to cancer cells that have not been contacted with the one or more glucocorticoid receptor agonists and the one or more inhibitors of calcineurin.

5. A method of inducing vacuolization of cancer cells in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin.

6. A method of inducing cell death of cancer cells, comprising administration to a subject (1) one or more glucocorticoid receptor agonists and (2) one or more inhibitors of calcineurin; wherein the number of cells undergoing cell death is increased compared to cancer cells that have not been contacted with the one or more glucocorticoid receptor agonists and the one or more inhibitors of calcineurin.

7. A method of inducing cell death of one or more cancer cells in vitro, comprising contacting the cancer cells with 1) one or more glucocorticoid receptor agonists and (2) one or more inhibitors of calcineurin; wherein the cancer cells are grown at about 100 percent or more confluence prior to contacting the cancer cells with the one or more glucocorticoid receptor agonists and one or more inhibitors of calcineurin.

8. The method of any one of claims 1-5, wherein the one or more glucocorticoid receptor agonists are selected from the group consisting of: a natural glucocorticoid receptor agonist, a synthetic glucocorticoid receptor agonist, a hydrocortisone-type glucocorticoid receptor agonist, a methasone-type glucocorticoid receptor agonist and an acetonide related glucocorticoid receptor agonist.

9. The method of any one of claims 1-8, wherein the glucocorticoid receptor agonist comprises hydrocortisone.

10. The method of any one of claims 1-9, wherein the glucocorticoid receptor agonist comprises dexamethasone.

11. The method of any one of claims 1-10, wherein the calcineurin inhibitor comprises a cyclosporin.

12. The method of claim 11, wherein the cyclosporin comprises cyclosporin A.

13. The method of any one of claims 1-12, wherein the one or more glucocorticoid receptor agonist comprises hydrocortisone and the more calcineurin inhibitor comprises cyclosporin A.

14. The method of any one of the above claims, wherein the one or more glucocorticoid receptor agonists and the one or more inhibitors of calcineurin are administered to the subject or contacted with the cancer cells concurrently.

15. The method of any one of claims 1-10, wherein the one or more glucocorticoid receptor agonists and the one or more inhibitors of calcineurin are administered to the subject or contacted with the cancer cells sequentially.

16. The method of claim 15, wherein the one or more glucocorticoid receptor agonists are administered to the subject or contacted with the cancer cells prior to the administration of or contact with the one or more inhibitors of calcineurin.

17. The method of claim 15, wherein the one or more glucocorticoid receptor agonists are administered to the subject or contacted with the cancer cells after administration of or contact with the one or more inhibitors of calcineurin.

18. The method of any one of the above claims, wherein the cancer cells overexpress MYC compared to an otherwise identical non-cancer cell.

19. The method of any one of claims 1-17, wherein the cancer cells overexpress MYC-Nick compared to an otherwise identical non-cancer cell.

20. The method of any one of the above claims, wherein the cancer cells express BRAF^{V600E}.

21. The method of claim 20, wherein the cancer cells exhibit loss of TP53 expression.

22. The method of any one of the above claims, wherein the cancer is metastatic.

23. The method of any one of the above claims, wherein the administration of or contacting with the one or more glucocorticoid receptor agonists and the one or more inhibitors of calcineurin results in a reduction in cancer proliferation, increased cancer cell death, or combinations thereof, compared to otherwise identical cancer cells not contacted with the one or more glucocorticoid receptor agonists and the one or more inhibitors of calcineurin.

24. The method of claim 23, wherein the administration of or contacting with the one or more glucocorticoid receptor agonists and the one or more inhibitors of calcineurin results in a 2-fold or greater reduction in cancer cell proliferation as compared to otherwise identical cancer cells not contacted with the one or more glucocorticoid receptor agonists and the one or more inhibitors of calcineurin.

25. The method of claim 23, wherein the administration of or contacting with the one or more glucocorticoid receptor agonists and the one or more inhibitors of calcineurin results in a 2-fold or greater increase in cancer cell death as compared to otherwise identical cancer cells not contacted with the one or more glucocorticoid receptor agonists and the one or more inhibitors of calcineurin.

26. The method of any one of claim 5, 23 or 25, wherein the cancer cell death is non-apoptotic cell death.

27. The method of claim 26, wherein the non-apoptotic cell death is characterized by an increase in the presence of vacuoles compared to cancer cells that have not been contacted with the one or more glucocorticoid receptor agonists and the one or more inhibitors of calcineurin.

28. The method of claim 27, wherein the increase in the presence of vacuoles is at least 50 fold or more greater than the presence of vacuoles in cancer cells treated with the one or more inhibitors of calcineurin alone.

29. The method of claim 27, wherein the increase in the presence of vacuoles is at least 5-fold or more greater than the presence of vacuoles in cancer cells treated with the one or more glucocorticoid receptor agonists alone.

30. The method of any one of claims 27-29, wherein the vacuoles have a maximum diameter of about 50 μm.

31. The method of any one of claims 27-30, wherein one or more of Calpain, Rab5, Rab11, LAMP1, MYC, MYC-Nick TFEB or ERM1 are localized to the vacuoles of the cells contacted with the one or more glucocorticoid receptor agonists and the one or more calcineurin inhibitors.

32. The method of any one of the above claims, wherein the cancer cells exhibit increased intracellular junctions as compared to cancer cells that have not been contacted with the one or more glucocorticoid receptor agonists and the one or more calcineurin inhibitors.

33. The method of any one of claims 5-32, wherein the cancer cells are grown in vitro.

34. The method of claim 33 wherein the cancer cells contacted with the one or more glucocorticoid receptor agonists and the one or more calcineurin inhibitors when grown to confluence, exhibit a greater area of cells growing in a single monolayer compared to an area of cells growing in a multi-layer, as compared to otherwise identical cancer cells grown to confluence and not treated with the one or more glucocorticoid receptor agonists and the one or more calcineurin inhibitors.

35. The method of claim 34, wherein greater than 50% of the area of the cancer cells contacted with the one or more glucocorticoid receptor agonists and the one or more calcineurin inhibitors and grown to confluence, exhibit growth in a monolayer.

36. The method of any one of claims 14-35, wherein the one or more glucocorticoid receptor agonists comprises hydrocortisone.

37. The method of any one of claims 14-36, wherein the one or more inhibitors of calcineurin comprises a cyclosporin.

38. The method of claim 37, wherein the cyclosporin comprises cyclosporin A.

39. The method of claim 37, wherein the one or more glucocorticoid receptor agonists comprises hydrocortisone and the one or more calcineurin inhibitors comprises cyclosporin A.

40. A method of screening for compounds that restore contact inhibition in a population of cancer cells, the method comprising:
(i) contacting the population of cancer cells growing on a tissue culture vessel with a test compound in vitro;
(ii) monitoring the viability and morphology of the cancer cells after the population of cells have grown to about 100% or more confluence to create a monolayer of cells on the bottom of the culture vessel;
(iii) identifying a test compound as a compound that restores contact inhibition when, compared to a distinct population of the cancer cells that has not been contacted with the test compound; wherein the population of cancer cells contacted with the test compound exhibits:
(a) a decrease in the number of cells growing as a multi-layer above the monolayer of the confluent cells,
(b) an increase in cell death of cells growing as a multi-layer above the monolayer of confluent cells;
(c) an increase in the number of cells with intercellular junctions, and
(d) an increase in cell death of cells that have reduced intercellular junctions as compared to normal cells.

41. The method of claim 40, wherein the population of cancer cells are contacted with the test compound when the population of cancer cells are grown to about 100% confluence or more.

42. The method of claim 40, wherein the population of cancer cells contacted with the test compound further exhibit reduced anchorage independent growth when grown in soft agar in vitro, as compared to the population of cells that has not been contacted with the test compound.

43. The method of claim 40, wherein the population of cancer cells contacted with the test compound further exhibits an increase in the area of monolayer growth compared to the area of multilayer growth relative to cells not contacted with the test compound.

44. The method of claim 40, wherein the population of cancer cells contacted with the test compound further exhibits an increase in the presence of vacuoles compared to the population of the cancer cells that has not been contacted with the test compound.

45. The method of claim 41, wherein the vacuoles have a maximum diameter of about 50 μm.

46. The method of claim 41 or 44, wherein one or more of Calpain, Rab5, Rab11, LAMP1, MYC, NYC-Nick, TFEB or ERM1 are localized to the vacuoles.

47. The method of any one of claims 40-45, wherein the cancer cells are a cancer cell line.

48. The method of any one of claims 40-47, wherein the cancer cells overexpress MYC compared to otherwise identical non-cancer cells.

49. The method of any one of claims 40-47, wherein the cancer cells overexpress MYC-Nick compared to otherwise identical non-cancer cells.

50. The method of any one of claims 40-48, wherein the cancer cells express BRAF^{V600E}.

51. The method of claim 50, wherein the cancer cells have a loss of TP53.

52. The method of any one of claims 40-50, wherein the increase in cell death is non-apoptotic cell death.

53. The method of any one of claims 40-52, wherein the intercellular junctions are selected from adherens junctions and tight junctions.

54. The method of any one of claims 40-53, wherein the cells growing above the monolayer of confluent cells exhibit increased phosphorylation of Ser10 of Histone 3 compared to the cells growing as a monolayer.

55. A method of inducing vacuolation of one or more cancer cells, comprising contacting the cancer cells with 1) one or more glucocorticoid receptor agonists and (2) one or more inhibitors of calcineurin; wherein the cancer cells, following contact with the one or more glucocorticoid receptor agonists and one or more inhibitors of calcineurin, are characterized by the presence of vacuoles; wherein the vacuoles:
(i) have a maximum diameter of about 50 μm; and
(ii) one or more of Calpain, Rab5, Rab11, LAMP1, MYC, MYC-Nick, TFEB or ERM1 are localized to the vacuoles.

56. A method of inducing non-apoptotic cell death of one or more cancer cells, comprising contacting the cancer cells with 1) one or more glucocorticoid receptor agonists and (2) one or more inhibitors of calcineurin; wherein the cancer cells, following contact with the one or more glucocorticoid receptor agonists and one or more inhibitors of calcineurin, are characterized by the presence of vacuoles; wherein the vacuoles:
(i) have a maximum diameter of about 50 μm;
(ii) one or more of Calpain, Rab5, Rab11, LAMP1, MYC, MYC-Nick, TFEB or ERM1 are localized to the vacuoles; and
(iii) comprise condensed and minimized nuclei at inner periphery of vacuoles.

57. The method of claim 55 or 56, wherein the one or more inhibitors of calcineurin comprises a cyclosporin.

58. The method of claim 57, wherein the cyclosporin comprises cyclosporin A.

59. The method of any one of claims 55-58, wherein the one or more glucocorticoid receptor agonists comprises hydrocortisone.

60. The method of any one of claims 55-59, wherein the one or more glucocorticoid receptor agonists comprises dexamethasone.

61. The method of claim 56, wherein the one or more glucocorticoid receptor agonists comprises hydrocortisone and the one or more calcineurin inhibitors comprises cyclosporin A.

62. The method of claim 61, wherein the cancer cells overexpress MYC-Nick compared to otherwise identical non-cancer cells.
